(19) United States
(12) Patent Application Publication
(10) Pub. No.: US 2010/0317656 A1
(43) Pub. Date: Dec. 16, 2010

(54) IMIDAZO[1,2-a]PYRIDINE-2-CARBOXAMIDE DERIVATIVES, PREPARATION THEREOF AND APPLICATION THEREOF IN THERAPEUTICS

(75) Inventor: Jean-Francois PEYRONEL, Palaiseau (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/828,372

(22) Filed: Jul. 1, 2010

Related U.S. Application Data
(63) Continuation of application No. PCT/FR2008/001835, filed on Dec. 31, 2008.

(30) Foreign Application Priority Data
Jan. 2, 2008 (FR) 0800004

(51) Int. Cl. A61K 31/541; C07D 471/04; A61K 31/437; C07D 413/14; A61K 31/5383; A61K 31/5377; C07D 417/06; A61P 25/00; A61P 25/28; A61P 25/24; A61P 19/10; A61P 29/00; A61P 35/00

(52) U.S. Cl. 514/228.2; 546/121; 514/300; 544/105; 514/230.5; 544/127; 514/233.2; 544/58.4

(57) ABSTRACT

Compounds of Formula (I):

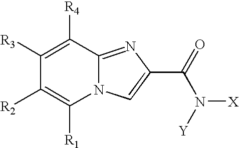

wherein X, Y, R₁, R₂, R₃, and R₄ are as defined in the disclosure, or a acid addition salt thereof; and therapeutic use thereof.

The present invention relates to imidazo[1,2-α]pyridine-2-carboxamide derivatives, to their preparation and to their therapeutic application in the treatment or prevention of diseases involving Nurr-1 nuclear receptors, also known as NR4A2, NOT, TINUR, RNR-1, and HZF3.

A subject-matter of the present invention is the compounds of formula (I):

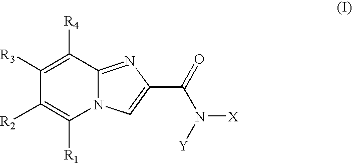

in which:
X and Y form, with the nitrogen atom which carries them, a saturated or partially saturated, mono- or bicyclic, 5 to 10-membered cyclic amine optionally comprising from 1 to 4 additional heteroatoms chosen from O, S or N which is optionally substituted by a halogen atom or a (C₁-C₆)alkyl, (C₁-C₆)alkoxy, cyano, NRaRb or COOR₈ group, the said (C₁-C₆)alkyl and (C₁-C₆)alkoxy groups optionally being substituted by one or more halogen atoms;
R₁ represents a hydrogen atom, a halogen atom or a (C₁-C₆)alkoxy, (C₁-C₆)alkyl or NRcRd group, it being possible for the alkyl and alkoxy groups optionally to be substituted by one or more halogen atoms or a hydroxyl, amino or (C₁-C₆)alkoxy group;
R₂ represents one of the following groups:
   a hydrogen atom,
   a (C₁-C₆)alkyl group optionally substituted by one or more groups chosen, independently of one another, from a hydroxyl, a halogen atom, an NRaRb group, a (C₁-C₆)alkoxy group or a phenyl group,
   a (C₁-C₆)alkoxy group optionally substituted by one or more groups chosen, independently of one another, from a hydroxyl, a halogen atom or an NRaRb group,
   a (C₂-C₆)alkenyl group,
   a (C₂-C₆)alkynyl group,
   a —CO—R₅ group,
   a —CO—NR₆R₇ group,
   a —CO—O—R₈ group,
   an —NR₉—CO—R₁₀ group,
   an —N═CH—NRaRb group,
   an NR₁₁R₁₂ group,
   a halogen atom,
   a cyano, nitro, hydroxyiminoalkyl or alkoxyiminoalkyl group,
   a (C₁-C₆)alkylthio group,
   a (C₁-C₆)alkylsulphinyl group,
   a (C₁-C₆)alkylsulphonyl group,
   a ((C₁-C₆)alkyl)₃silylethynyl group,
   an —SO₂—NR₉R₁₀ group,
   a phenyl group optionally substituted by one or more groups chosen, independently of one another, from the following atoms or groups: halogen, (C₁-C₆)alkoxy, cyano, NRaRb, —CO—R₅, —CO—NR₆R₇, —CO—O—R₈ or (C₁-C₆)alkyl optionally substituted by one or more hydroxyl or NRaRb groups,
   a heterocyclic group optionally substituted by a halogen atom or a (C₁-C₆)alkyl, (C₁-C₆)alkoxy, cyano, NRaRb or COOR₈ group, the said (C₁-C₆)alkyl and (C₁-C₆)alkoxy groups optionally being substituted by one or more halogen atoms or hydroxyl groups;
R₃ represents a hydrogen atom, a (C₂-C₆)alkyl group, a (C₁-C₆)alkoxy group or a halogen atom;
R₄ represents a hydrogen atom, a (C₁-C₄)alkyl group, a (C₁-C₄)alkoxy group or a fluorine atom;
R₅ represents a hydrogen atom, a phenyl group or a (C₁-C₆)alkyl group;
R₆ and R₇, which are identical or different, represent a hydrogen atom or a (C₁-C₆)alkyl group or form, with the nitrogen atom which carries them, a 4- to 7-membered ring optionally including another heteroatom chosen from N, O or S;
R₈ represents a (C₁-C₆)alkyl group;
R₉ and R₁₀, which are identical or different, represent a hydrogen atom or a (C₁-C₆)alkyl group;
R₁₁ and R₁₂, which are identical or different, represent a hydrogen atom or a (C₁-C₆)alkyl group;
Ra and Rb are, independently of one another, hydrogen or (C₁-C₆)alkyl or form, with the nitrogen atom which carries them, a 4- to 7-membered ring;
Rc is hydrogen and Rd is (C₁-C₆)alkyl;
with the exception of 2-(2,3-dihydro-1H-indol-1-ylcarbonyl)-5-methylimidazo[1,2-α]pyridine and 2-(4-thiomorpholin-1-ylcarbonyl)-6-chloroimidazo[1,2-α]pyridine, in the form of the base or of an addition salt with an acid.

The compounds 2-(2,3-dihydro-1H-indol-1-ylcarbonyl)-5-methylimidazo[1,2-α]pyridine and 2-(4-thiomorpholin-1-ylcarbonyl)-6-chloroimidazo[1,2-α]pyridine are respectively cited in databases under the numbers RN=878113-13-4 and RN=951974-53-1. No pharmacological or therapeutic activity is demonstrated for these compounds. They have been specifically excluded from the formula (I) according to the present invention.

Furthermore, the document US 2006/0211747 teaches a method for identifying compounds which are Cdc34 inhibitors, one of the compounds identified being an imidazo[1,2-α]pyridine derivative not included in the formula (I) according to the present invention.

The following nomenclatures are regarded as equivalents:
2-(2,3-dihydro-1H-indol-1-ylcarbonyl)-5-methylimidazo[1,2-α]pyridine and (2,3-dihydro-1H-indol-1-yl)(5-methylimidazo[1,2-α]pyridin-2-yl)methanone, and
2-(4-thiomorpholin-1-ylcarbonyl)-6-chloroimidazo[1,2-α]-pyridine, and (6-chloroimidazo[1,2-α]pyridin-2-yl)(4-thiomorpholinyl)methanone.

The compounds of formula (I) can comprise one or more asymmetric carbon atoms. They can therefore exist in the form of enantiomers or diastereoisomers. These enantiomers or diastereoisomers and their mixtures, including racemic mixtures, come within the invention.

The compounds of formula (I) can exist in the form of bases or of addition salts with acids. Such addition salts come within the invention.

These salts can be prepared with pharmaceutically acceptable acids but the salts of other acids, for example of use in the purification or the isolation of the compounds of formula (I), also come within the invention.

The compounds of formula (I) can also exist in the form of hydrates or solvates, namely in the form of combinations or associations with one or more molecules of water or with a solvent. Such hydrates and solvates also come within the invention.

In the context of the present invention:
a halogen atom is understood to mean a fluorine, a chlorine, a bromine or an iodine;
an alkyl group is understood to mean a saturated, linear, branched or cyclic, aliphatic group which is optionally substituted by a saturated, linear, branched or cyclic, alkyl group. Mention may be made, by way of examples, of the methyl, ethyl, propyl, isopropyl, butyl, isobutyl, tert-butyl, cyclopropyl, cyclobutyl, cyclopentyl, cyclohexyl or methylcyclopropyl groups, and the like;
an alkenyl group is understood to mean a mono- or polyunsaturated, linear or branched, aliphatic group comprising, for example, one or two ethylenic unsaturations;
an alkoxy group is understood to mean an —O-alkyl radical where the alkyl group is as defined above;
an alkynyl group is understood to mean a mono- or polyunsaturated, linear or branched, aliphatic group comprising, for example, one or two ethylynic unsaturations;
a cyclic amine is understood to mean a mono- or bicyclic group comprising from 5 to 10 ring members and comprising at least one nitrogen atom which can additionally comprise from 1 to 4 heteroatoms chosen from N, O and S, it being possible for this group to be saturated or partially saturated. Mention may be made, as examples of cyclic amines, of benzoxazine, indoline, isoindoline, tetrahydroisoquinoline, morpholine, piperidine, pyrrolidine, pyrroline, tetrahydropyridine, tetrahydroquinoline, thiomorpholine, dihydroxybenzoxazine or tetrahydrothienopyridine;
a heterocyclic group is understood to mean an aromatic, unsaturated, saturated or partially saturated and mono- or bicyclic group comprising from 5 to 10 atoms, including from 1 to 4 heteroatoms chosen from N, O and S. Mention may be made, as examples of heterocyclic groups, of: pyrrole, furan, thiophene, pyrazole, imidazole, triazole, tetrazole, oxazole, isoxazole, oxadiazole, thiazole, isothiazole, thiadiazole, pyridine, pyrimidine, pyrazine, pyridazine, triazine, furofuran, thienothiophene, pyrrolopyrrole, pyrroloimidazole, pyrrolopyrazole, pyrrolotriazole, imidazoimidazole, imidazopyrazole, furopyrrole, furoimidazole, furopyrazole, furotriazole, pyrrolooxazole, imidazooxazole, pyrazolooxazole, furooxazole, oxazolooxazole, oxazoloisoxazole, pyrroloisoxazole, imidazoisoxazole, pyrazoloisoxazole, isoxazoloisoxazole, furoisoxazole, isoxazolooxadiazole, pyrrolooxadiazole, furooxadiazole, isoxazolooxadiazole, thienopyrrole, thienoimidazole, thienopyrazole, thienotriazole, pyrrolothiazole, imidazothiazole, pyrazolothiazole, triazolothiazole, furothiazole, oxazolothiazole, oxazoloisothiazole, pyrroloisothiazole, imidazoisothiazole, pyrazoloisothiazole, isoxazoloisothiazole, furoisothiazole, pyrrolothiadiazole, imidazothiadiazole, furothiadiazole, isoxazolothiadiazole, oxazolothiadiazole, isothiazolothiadiazole, indole, isoindole, benzimidazole, indazole, indolizine, benzofuran, isobenzofuran, benzothiophene, benzo[c]thiophene, pyrrolopyridine, imidazopyridine, pyrazolopyridine, triazolopyridine, tetrazolopyridine, pyrrolopyrimidine, imidazopyrimidine, pyrazolopyrimidine, pyrrolopyrazine, imidazopyrazine, pyrazolopyrazine, pyrrolopyridazine, imidazopyridazine, pyrazolopyridazine, triazolopyridazine, pyrrolotriazine, furopyridine, furopyrimidine, furopyrazine, furopyridazine, furotriazine, oxazolopyridine, oxazolopyrimidine, oxazolopyrazine, oxazolopyridazine, isoxazolopyridine, isoxazolopyrimidine, isoxazolopyrazine, isoxazolopyridazine, oxadiazolopyridine, benzoxazole, benzisoxazole, benzoxadiazole, thienopyridine, thienopyrimidine, thienopyrazine, thienopyridazine, thienotriazine, thiazolopyridine, thiazolopyrimidine, thiazolopyrazine, thiazolopyridazine, isothiazolopyridine, isothiazolopyrimidine, isothiazolopyrazine, isothiazolopyridazine, thiadiazolopyridine, thiadiazolopyrimidine, benzothiazole, benzoisothiazole, benzothiadiazole, quinoline, isoquinoline, cinnoline, phthalazine, quinoxaline, quinazoline, naphthyridine, benzotriazine, pyridopyrimidine, pyridopyrazine, pyridopyridazine, pyridotriazine, pyrimidopyrimidine, pyrimidopyrazine, pyrimidopyridazine, pyrazinopyrazine, pyrazinopyridazine, pyrazinotriazine or pyridazinopyridazine. These groups can exist in the saturated or partially saturated form.

Various subsets of compounds are defined below, which also come within the invention.

Among the compounds of general formula (I) as defined above, a first group of compounds is composed of the compounds for which R₂ is other than a hydrogen or chlorine atom and the other substituents are as defined above, in the form of the base or of an addition salt with an acid.

Among the compounds of general formula (I) as defined above, a second group of compounds is composed of the compounds for which R₂ represents one of the following groups:
a (C₁-C₆)alkoxy group,
an NR₁₁R₁₂ group,
a phenyl group optionally substituted by a —CO—R₅, or (C₁-C₆)alkyl group, itself optionally substituted by a hydroxyl;
a heterocyclic group optionally substituted by an NRaRb or (C₁-C₆)alkyl group, itself optionally substituted by a hydroxyl;
R₅ represents a (C₁-C₆)alkyl group,
R₁₁ and R₁₂, which are identical or different, represent a hydrogen atom or a (C₁-C₆)alkyl group;
Ra and Rb are, independently of one another, hydrogen or (C₁-C₆)alkyl; and the other substituents are as defined above, in the form of the base or of an addition salt with an acid.

Among the compounds of general formula (I) as defined above, a third group of compounds is composed of the compounds for which R₂ represents one of the following groups:
a (C₁-C₆)alkoxy group,
an NR₁₁R₁₂ group,
a phenyl group optionally substituted by a —CO—R₅ or (C₁-C₆)alkyl group, itself optionally substituted by a hydroxyl,
a pyridyl group, a pyrazolyl group, a furyl group, an oxazolyl group, a triazolyl group, a pyrrolyl group or an imidazolyl group optionally substituted by an NRaRb or (C₁-C₆)alkyl group, itself optionally substituted by a hydroxyl;
R₅ represents a (C₁-C₆)alkyl group,
R₁₁ and R₁₂, which are identical or different, represent a hydrogen atom or a (C₁-C₆)alkyl group;
   Ra and Rb are, independently of one another, hydrogen or (C₁-C₆)alkyl;
and the other substituents are as defined above, in the form of the base or of an addition salt with an acid.

Among the compounds of general formula (I) as defined above, a fourth group of compounds is composed of the compounds for which R₂ represents one of the following groups:
a (C₁-C₆)alkoxy group,
an NR₁₁R₁₂ group,
a phenyl group optionally substitute by a —CO—R₅ or (C₁-C₆)alkyl group, itself optionally substituted by a hydroxyl;
a pyridyl group optionally substituted by an amino group, a pyrazolyl group, a furyl group optionally substituted by a hydroxylmethyl group, an oxazolyl group, a triazolyl group, a pyrrolyl group or an imidazolyl group;
R₅ represents a (C₁-C₆)alkyl group,
R₁₁ and R₁₂, which are identical or different, represent a hydrogen atom or a (C₁-C₆)alkyl group;
and the other substituents are as defined above, in the form of the base or of an addition salt with an acid.

Among the compounds of general formula (I) as defined above, a fifth group of compounds is composed of the compounds for which X and Y form, with the nitrogen atom which carries them, a saturated or partially saturated, mono- or bicyclic, 5- to 10-membered cyclic amine optionally comprising an additional heteroatom chosen from O or S and optionally substituted by a group chosen from a halogen atom,

and the other substituents are as defined above, in the form of the base or an addition salt with an acid.

Among the compounds of general formula (I) as defined above, a sixth group of compounds is composed of the compounds for which -NKY represents a dihydrobenzoxazine, indoline, isoindoline, morpholine, piperidine, pyrrolidine, pyrroline, tetrahydropyridine, tetrahydroquinoline, thiomorpholine or tetrahydrothienopyridine group optionally substituted by one or more halogen atoms;

and the other substituents are as defined above, in the form of the base or of an addition salt with an acid.

Among the compounds of general formula (I) as defined above, a seventh group of compounds is composed of the compounds for which:

-NKY represents a dihydrobenzoxazine, indoline, isoindoline, morpholine, piperidine, pyrrolidine, pyrroline, tetrahydropyridine, tetrahydroquinoline, thiomorpholine or tetrahydrothienopyridine group optionally substituted by a halogen atom;
R₂ represents one of the following groups:
a (C₁-C₆)alkoxy group,
an NR₁₁R₁₂ group,
a phenyl group optionally substituted by a group chosen from a halogen, a (C₁-C₆)alkoxy group or a (C₁-C₆)alkyl group optionally substituted by a hydroxyl or a C(O)R₅ group,
a pyridyl group, a pyrazolyl group, a furyl group, an oxazolyl group, a triazolyl group, a pyrrolyl group or an imidazolyl group optionally substituted by an NRaRb or (C₁-C₆)alkyl group, itself optionally substituted by a hydroxyl;
R₅ represents a (C₁-C₆)alkyl group,
R₁₁ and R₁₂, which are identical or different, represent a hydrogen atom or a (C₁-C₆)alkyl group;
   Ra and Rb are, independently of one another, hydrogen or (C₁-C₆)alkyl;
R₁, R₃ and R₄ represent a hydrogen atom, in the form of the base or of an addition salt with an acid. Among the compounds of general formula (I) as defined above, an eighth group of compounds is composed of the compounds for which:

-NKY represents a dihydrobenzoxazine group, an indoline group optionally substituted by a fluorine atom, an isoindoline group, a morpholine group, a piperidine group, a pyrrolidine group, a pyrroline group, a tetrahydropyridine group, a tetrahydroquinoline group, a thiomorpholine group or a tetrahydrothienopyridine group;

R₂ represents a methoxy group, a phenyl group substituted by a hydroxymethyl, hydroxyethyl, hydroxymethylethyl, acetyl or N-dimethyl group, a pyridyl group optionally substituted by an amino group, a pyrazolyl group, a furyl group optionally substituted by a hydroxymethyl group, an oxazolyl group, a triazolyl group, a pyrrolyl group or an imidazoyl group; R₁, R₃ and R₄ represent a hydrogen atom, in the form of the base or of an addition salt with an acid.

Among the compounds of general formula (I) as defined above, a ninth group of compounds is composed of the compounds for which NXY represents a dihydrobenzoxazine, indoline, isoindoline, tetrahydroisoquinoline, morpholine, piperidine, pyrrolidine, pyrroline, tetrahydropyridine, tetrahydroquinoline, thiomorpholine or tetrahydrothienopyridine group, these groups optionally being substituted by a fluorine atom;
R₁, R₃ and R₄ represent a hydrogen atom;
R₂ represents a methoxy group or a phenyl group substituted by a hydroxymethyl or N-dimethyl group, in the form of the base or of an addition salt with an acid.

Mention may in particular be made, among the compounds of formula (I) which are subject-matters of the invention, of the following compounds:
{3-[2-(Piperidin-1-ylcarbonyl)imidazo[1,2-α]pyridin-6-yl]phenyl}methanol
{3-[2-(2,3-Dihydro-4H-1,4-benzoxazin-4-ylcarbonyl)imidazo[1,2-α]pyridin-6-yl]phenyl}methanol
{3-[2-(Morpholin-4-ylcarbonyl)imidazo[1,2-α]pyridin-6-yl]phenyl}methanol
2-(1,2,5,6-Tetrahydropyridin-1-yl)carbonyl-N,N-dimethylimidazo[1,2-α]pyridin-6-amine
{3-[2-(2,3-Dihydro-1H-indol-1-ylcarbonyl)imidazo[1,2-α]pyridin-6-yl]phenyl}methanol
(3-{2-[(6-Fluoro-2,3-dihydro-1H-indol-1-yl)carbonyl]imidazo[1,2-α]pyridin-6-yl}phenyl)methanol
{3-[2-(2,5-Dihydro-1H-pyrrol-1-ylcarbonyl)imidazo[1,2-α]pyridin-6-yl]phenyl}methanol
{3-[2-[(1,2,5,6-Tetrahydropyridin-1-yl)carbonyl]imidazo[1,2-α]pyridin-6-yl]phenyl}methanol
{3-[2-(Pyrrolidin-1-ylcarbonyl)imidazo[1,2-α]pyridin-6-yl]phenyl}methanol
{3-[2-(4,5,6,7-Tetrahydrothieno[3,2-α]pyridin-5-ylcarbonyl)imidazo[1,2-α]pyridin-6-yl]phenyl}methanol
2-(2,3-Dihydro-4H-1,4-benzoxazin-4-ylcarbonyl)-N,N-dimethylimidazo[1,2-α]pyridin-6-amine
{3-[2-(1,2,3,4-Tetrahydroquino line-1-ylcarbonyl)imidazo[1,2-α]pyridin-6-yl]phenyl}methanol
{3-[2-(1,3-Dihydro-2H-isoindol-2-ylcarbonyl)imidazo[1,2-α]pyridin-6-yl]phenyl}methanol
2-(2,3-Dihydro-1H-indol-1-ylcarbonyl)-N,N-dimethylimidazo[1,2-α]pyridin-6-amine
2-(1,3-Dihydro-2H-isoindol-2-ylcarbonyl)-N,N-dimethylimidazo[1,2-α]pyridin-6-amine
N,N-Dimethyl-2-(piperidin-1-ylcarbonyl)imidazo[1,2-α]pyridin-6-amine
2-[(6-Fluoro-2,3-dihydro-1H-indol-1-yl)carbonyl]-N,N-dimethylimidazo[1,2-α]pyridin-6-amine
N,N-Dimethyl-2-(morpholin-4-ylcarbonyl)imidazo[1,2-α]pyridin-6-amine
2-(2,5-Dihydro-1H-pyrrol-1-ylcarbonyl)-N,N-dimethylimidazo[1,2-α]pyridin-6-amine
{3-[2-(Thiomorpholin-4-ylcarbonyl)imidazo[1,2-α]pyridin-6-yl]phenyl}methanol
N,N-Dimethyl-2-(pyrrolidin-1-ylcarbonyl)imidazo[1,2-α]pyridin-6-amine
2-(4,5,6,7-Tetrahydrothieno[3,2-c]pyridin-5-ylcarbonyl)-N,N-dimethylimidazo[1,2-α]pyridin-6-amine
N,N-Dimethyl-2-(thiomorpholin-4-ylcarbonyl)imidazo[1,2-α]pyridin-6-amine
2-(1,2,5,6-Tetrahydropyridin-1-yl)carbonyl-6-methoxy-5-methylimidazo[1,2-α]pyridine
6-(Pyridin-2-yl)-2-[(1,2,5,6-tetrahydropyridin-1-yl)carbonyl]imidazo[1,2-α]pyridine
2-(2,3-Dihydro-1H-indol-1-ylcarbonyl)-6-(pyridin-2-yl)imidazo[1,2-α]pyridine
2-(2,3-Dihydro-1H-indol-1-ylcarbonyl)-6-(1H-pyrazol-3-yl)imidazo[1,2-α]pyridine
6-(Furan-2-yl)-2-[(1,2,5,6-tetrahydropyridin-1-yl)carbonyl]imidazo[1,2-α]pyridine
2-(2,3-Dihydro-1H-indol-1-ylcarbonyl)-6-(furan-2-yl)imidazo[1,2-α]pyridine
6-(Oxazol-2-yl)-2-[(1,2,5,6-tetrahydropyridin-1-yl)carbonyl]imidazo[1,2-α]pyridine
6-(Furan-3-yl)-2-[(1,2,5,6-tetrahydropyridin-1-yl)carbonyl]imidazo[1,2-α]pyridine
2-(2,3-Dihydro-1H-indol-1-ylcarbonyl)-6-(furan-3-yl)imidazo[1,2-α]pyridine
6-[5-(Hydroxymethyl)furan-2-yl]-2-[(1,2,5,6-tetrahydropyridin-1-yl)carbonyl]imidazo[1,2-α]pyridine
[(1,2,5,6-Tetrahydropyridin-1-yl)carbonyl]-6-(1H-1,2,4-triazol-3-yl)imidazo[1,2-α]pyridine
2-(2,3-Dihydro-1H-indol-1-ylcarbonyl)-6-(1H-1,2,4-triazol-3-yl)imidazo[1,2-α]pyridine
6-(6-Aminopyridin-2-yl)-2-[(1,2,5,6-tetrahydropyridin-1-yl)carbonyl]imidazo[1,2-α]pyridine and its dihydrochloride
6-(6-Aminopyridin-2-yl)-2-(2,3-Dihydro-1H-indol-1-ylcarbonyl)imidazo[1,2-α]pyridine and its dihydrochloride
6-(1H-Pyrrol-3-yl)-2-[(1,2,5,6-tetrahydropyridin-1-yl)carbonyl]imidazo[1,2-α]pyridine
2-(2,3-Dihydro-1H-indol-1-ylcarbonyl)-6-(1H-pyrrol-3-yl)imidazo[1,2-α]pyridine
2-(2,3-Dihydro-1H-indol-1-ylcarbonyl)-6-(furan-2-yl)imidazo[1,2-α]pyridine and its trifluoroacetate(1.1)
2-(2,3-Dihydro-1H-indol-1-ylcarbonyl)-6-[5-(hydroxymethyl)furan-2-yl]imidazo[1,2-α]pyridine and its trifluoroacetate(1•1)
6-(1H-Imidazol-4-yl)-2-[(1,2,5,6-tetrahydropyridin-1-yl)carbonyl]imidazo[1,2-α]pyridine
2-(2,3-Dihydro-1H-indol-1-ylcarbonyl)-6-(1H-imidazol-4-yl)imidazo[1,2-α]pyridine
2-[(1,2,5,6-Tetrahydropyridin-1-yl)carbonyl]-6-(1H-1,2,3-triazol-4-yl)imidazo[1,2-α]pyridine
2-(2,3-Dihydro-1H-indol-1-ylcarbonyl)-6-(1H-1,2,3-triazol-4-yl)imidazo[1,2-α]pyridine
6-(3-Acetylphenyl)-2-[(1,2,5,6-tetrahydropyridin-1-yl)carbonyl]imidazo[1,2-α]pyridine
(RS)-6-[3-(1-Hydroxyethyl)phenyl]-2-[(1,2,5,6-tetrahydropyridin-1-yl)carbonyl]imidazo[1,2-α]pyridine
6-[3-(1-Hydroxy-1-methylethyl)phenyl]-2-[(1,2,5,6-tetrahydropyridin-1-yl)carbonyl]imidazo[1,2-α]pyridine
   and their addition salts with an acid.

In accordance with the invention, the compounds of general formula (I) can be prepared according to the process described in Scheme 1.

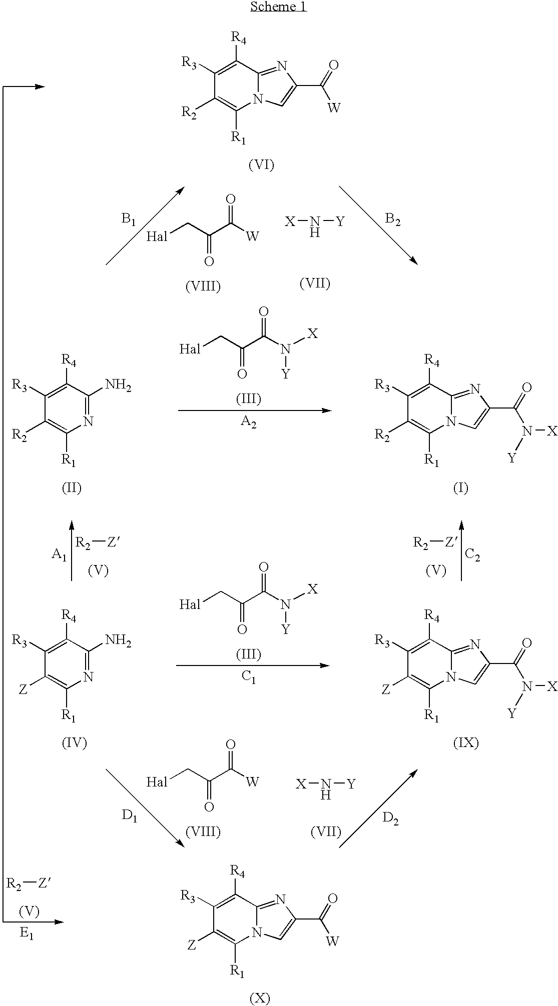

The first synthetic route (transformation A₂) consists in preparing, according to methods known to a person skilled in the art, a 2-aminopyridine of formula (II), in which R₁, R₂, R₃ and R₄ are defined as above, and in then forming the imidazo[1,2-α]pyridine ring by condensation with a halogenated derivative of 2-oxopropionamide (III), in which Hal represents a chlorine, bromine or iodine atom and X and Y are defined as above, by analogy with the methods described by J-J. Bourguignon et al. in Aust. J. Chem., 50, 719 (1997), and by J. G. Lombardino in J. Org. Chem., 30, 2403 (1965), for example.

The halogenated derivatives of 2-oxopropionamide (III) can be obtained, for example, according to the method described by R. Kluger et al. in J. Am. Chem. Soc., 106, 4017 (1984).

The 2-aminopyridines of formula (II), in which R₁, R₂, R₃ and R₄ are defined as above, can be prepared, for example, by the transformation A₁, that is to say:
by a coupling reaction of a 2-aminopyridine derivative of formula (IV), in which R₁, R₃ and R₄ are defined as above and Z represents a boryl, stannyl or silyl group, and of a derivative R₂-Z′ (V), in which R₂ is defined as above and Z′ represents a halogen atom, such as bromine or iodine, or a sulphonyloxy group,
by a coupling reaction of a 2-aminopyridine derivative of formula (IV), in which R₁, R₃ and R₄ are defined as above and Z represents a halogen atom, such as bromine or iodine, with a derivative R₂-Z′ (V), in which R₂ is defined as above and Z′ represents a reactive group, such as a boryl, stannyl or silyl group, or a hydrogen atom,
   or by any other method known to a person skilled in the art.

The second synthetic route (transformation B₂) consists in coupling an imidazopyridine-2-carboxylic acid or one of its derivatives of formula (VI), in which R₁, R₂, R₃ and R₄ are defined as above and W represents a hydroxyl group, a halogen atom or a (C₁-C₆)alkoxy group, with a cyclic amine X—NH—Y of formula (VII), in which X and Y are defined as above, according to methods known to a person skilled in the art. Thus, the acid can be converted beforehand to one of its reactive derivatives, such as acid halide, anhydride, mixed anhydride or activated ester, and then reacted with the amine (VII) in the presence of a base, such as diisopropylethylamine, triethylamine or pyridine, in an inert solvent, such as THF, DMF or dichloromethane. The coupling can also be carried out in the presence of a coupling agent, such as CDI, EDCI, HATU or HBTU, under the same conditions without isolation of reactive intermediate. Alternatively, the amine (VII) can be reacted with an ester of the acid of formula (VI) in the presence of a catalyst, such as trimethylaluminium according to the method of Weinreb, S. et al. (Tet. Lett. (1977), 18, 4171) or zirconium tert-butoxide.

The derivatives of the imidazopyridine-2-carboxylic acids of formula (VI), in which R₁, R₂, R₃ and R₄ are defined as above and W is (C₁-C₆)alkoxy, hydroxy or halogen, are prepared by condensation of a 2-aminopyridine of formula (II), in which R₁, R₂, R₃ and R₄ are defined as above, with a 3-halo-2-oxopropionic acid ester of formula (VIII), in which Hal represents a halogen and W is (C₁-C₆)alkoxy, under conditions similar to those used for the condensation of a derivative of formula (II) with a derivative of formula (III), followed, if appropriate, by the conversion of the ester to the acid and then to the acid chloride or other reactive derivative (transformation B₁).

The third synthetic route (transformation C₂) consists in coupling a derivative of general formula (IX), in which R₁, R₃, R₄, X and Y are defined as above and Z represents a halogen atom, such as bromine or iodine, a sulphonyloxy group or a reactive group, such as boryl, stannyl or silyl, to a derivative of formula R₂-Z′ (V), in which R₂ is defined as above and
Z′ represents a reactive group, such as a boryl, stannyl or silyl group, or a hydrogen atom, when Z represents a halogen atom or a sulphonyloxy group, or
Z′ represents a halogen atom, such as bromine or iodine, when Z represents a reactive group, such as a boryl, stannyl or silyl group, or a hydrogen atom.

The derivatives of general formula (IX), in which R₁, R₃, R₄, X, Y and Z are defined as above, can be prepared:
by condensation of a 2-aminopyridine of formula (IV), in which R₁, R₃, R₄ and Z are defined as above, with a 2-oxopropionamide derivative (III), in which Hal represents a chlorine, bromine or iodine atom and X and Y are defined as above, (transformation C₁) according to methods mentioned for the conversion of a compound of formula (II) to a compound of formula (I), or
by amidation of an imidazopyridine-2-carboxylic acid or one of its derivatives of formula (X), in which R₁, R₂, R₃ and R₄ are defined as above and W represents a hydroxyl group, a halogen atom or a (C₁-C₆)alkoxy group, with an amine X—NH—Y (VII), in which X and Y are defined as above, (transformation D₂) according to methods cited for the conversion of a compound of formula (VI) to a compound of formula (I).

The imidazopyridine-2-carboxylic acids or their derivatives of formula (X), in which R₁, R₃ and R₄ are defined as above, W is (C₁-C₆)alkoxy, hydroxyl or halogen and Z represents a boryl, stannyl or silyl group or a halogen atom, can be prepared (transformation D₁) by condensation of a 2-aminopyridine of formula (IV), in which R₁, R₃ and R₄ are defined as above and Z represents a boryl, stannyl or silyl group or a halogen atom, with a 3-halo-2-oxopropionic acid ester of formula (VIII), in which Hal represents a halogen and W is a (C₁-C₆)alkoxy group, under conditions similar to those mentioned above for the condensation of the 2-aminopyridines of formula (II) with a derivative of formula (VIII), in order to obtain the imidazopyridine-2-carboxylic acids or their derivatives of formula (VI) according to transformation B₁, followed, if appropriate, by the conversion of the ester to the acid and then to the acid chloride or other reactive derivative.

The imidazopyridine-2-carboxylic acids or their derivatives of formula (VI), in which R₁, R₂, R₃ and R₄ are defined as above and W is (C₁-C₆)alkoxy, hydroxyl or halogen, can also be prepared (transformation E₁) by coupling a derivative of general formula (X), in which R₁, R₃, and R₄ are defined as above, W is (C₁-C₆)alkoxy and Z represents a halogen atom, such as bromine or iodine, a sulphonyloxy group or a reactive group, such as boryl, stannyl or silyl, to a derivative of formula R₂-Z′ (V), in which R₂ is defined as above and
Z′ represents a reactive group, such as a boryl, stannyl or silyl group, or a hydrogen atom, when Z represents a halogen atom or a sulphonyloxy group, or
Z′ represents a halogen atom, such as bromine or iodine, when Z represents a reactive group, such as a boryl, stannyl or silyl group, or a hydrogen atom,
   followed, if appropriate, by the conversion of the ester to the acid and then to the acid chloride or other reactive derivative.

The couplings of the derivatives of formula (IV), (IX) or (X) with the products of formula (V) can be carried out by any method known to a person skilled in the art, in particular by operating in the presence of copper- or palladium-based catalysts, of ligands, such as phosphines, according to or by analogy with the methods described, for example, in the references which follow and references cited:
for reactions of Suzuki type: N. Miyaura and A. Suzuki, Chem. Rev., 95, 2457 (1995),
for reactions of Stille type: V. Farina et al., Org. React., 50, 1 (1997),
for reactions of Hiyama type: T. Hiyama et al., Top. Curr. Chem., 2002, 219, 61 (2002),
for reactions of Negishi type: E. Negishi et al., Chem. Rev., 103, 1979 (2003),
for reactions of Bellina type: M. Miura et al., Chem. Lett., 200 (2007).

It is also possible, in order to carry out the coupling, to form organometallic derivatives, such as zinc derivatives, as intermediates but without isolating them.

In accordance with the invention, it is also possible to prepare the compounds of general formula (I), (VI) and (II) in which R₂ is a heterocycle according to the processes described in Scheme 2.

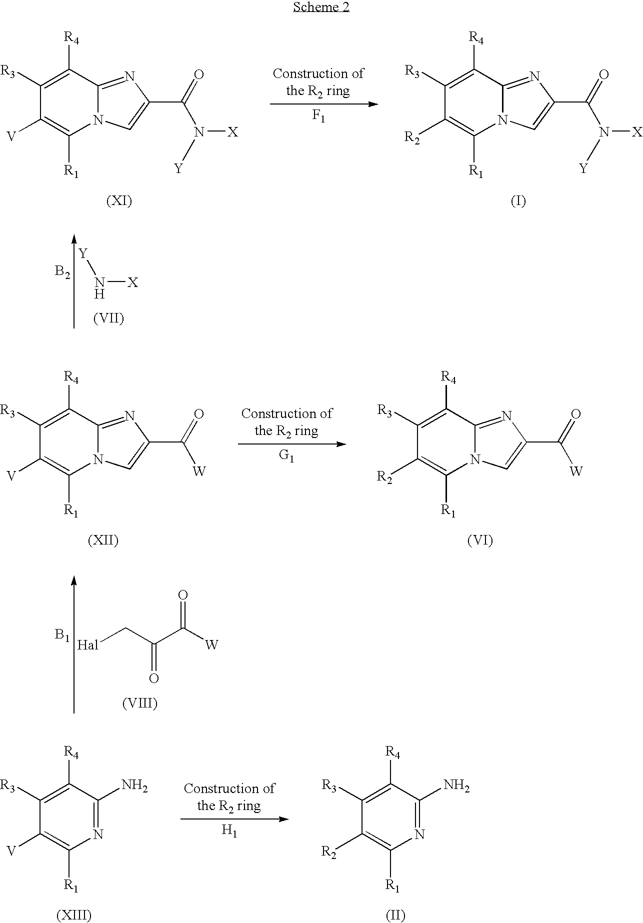

This synthetic route consists of the conversion of a compound of general formula (XI), (XII) or (XIII), in which R₁, R₃, R₄, X, Y and W are defined as above and V represents a precursor group which makes possible the construction of the heterocycle of formula R₂, according to methods known to a person skilled in the art.

By way of example, V can represent:
a 2-haloacyl group, such as bromoacetyl, or a 1-halo-2-oxoalkyl group, such as 1-bromo-2-oxoethyl, which can be converted, for example, to a thiazolyl, imidazolyl or oxazolyl group by treatment with thiourea, thioamide, guanidine, urea or amide derivatives,
an alkynyl group, such as ethynyl, which can be converted to a 1,2,3-triazol-4-yl group,
an acyl group, such as formyl, which can be converted, for example, to a 1,3-dioxolan-2-yl or oxazolyl group,
a cyano group, which can be converted, for example, to a dihydroimidazolyl(2) or 1,3,4-triazol-2-yl group.

The compounds of general formula (XI) can be obtained from the compounds of formula (XII), under the conditions described for the preparation of the compounds (I) from the imidazopyridine-2-carboxylic acid derivatives of formula (VI) by the transformations B₂.

The imidazopyridine-2-carboxylic acid derivatives of general formula (XII) can be obtained from the aminopyridines of formula (XIII), under the conditions described for the conversion of the aminopyridines of formula (II) to compounds of general formula (I) by the transformation A₂.

The products of formula (I) and their precursors of formula (II) or (IV) can be subjected, if desired and necessary, in order to obtain products of formula (I) or to be converted to other products of formula (I), to one or more of the following transformation reactions, in any order:
a) a reaction for the esterification or amidation of an acid functional group,
b) a reaction for the hydrolysis of an ester functional group to give an acid functional group,
c) a reaction for the amidation of an amine functional group,
d) a reaction for the transformation of a hydroxyl functional group to an alkoxy functional group,
e) a reaction for the oxidation of an alcohol functional group to give an aldehyde or ketone functional group,
f) a reaction for the transformation of aldehyde or ketone functional groups to give an alcohol functional group by reduction or by the action of an organometallic compound, such as an organomagnesium compound,
g) a reaction for the conversion of aldehyde or ketone functional groups to give an oxime derivative,
h) a reaction for the transformation of a nitrile radical to give an aldehyde functional group,
i) a reaction for the transformation of a nitrile radical to give a ketone functional group,
j) a reaction for the oxidation of an alkenyl group to give an aldehyde or ketone functional group,
k) a reaction for the olefination of an aldehyde or ketone functional group to give an alkenyl group,
l) a reaction for the dehydration of a hydroxyalkyl group to give an alkenyl group,
m) a reaction for the total or partial hydrogenation of an alkenyl or alkynyl group to give an alkenyl or alkyl group,
n) a reaction for the catalytic coupling of an organometallic derivative, such as a boron, tin or silicon derivative, with a halogenated derivative in order to introduce an alkyl, alkenyl, alkynyl, aryl or hetero aryl substituent,
o) a reaction for the reduction of a nitro group to give a primary amino group,
p) a reaction for the conversion of a primary or secondary amino group to a secondary or tertiary amino group by reductive amination or alkylation,
q) a reaction for the conversion of a primary amino group to an amidine group,
r) a reaction for the protection of the reactive functional groups,
s) a reaction for the removal of the protective groups which the protected reactive functional groups may carry,
t) a reaction for salification by an inorganic or organic acid or by a base in order to obtain the corresponding salt,
u) a reaction for the resolution of the racemic forms to give enantiomers, the said products of formula (I) thus obtained being, if appropriate, in all the possible isomeric forms, racemic, enantiomeric, diastereoisomeric and tautomeric.

In Schemes 1 and 2, the starting compounds and the reactants, when their method of preparation is not described, are commercially available or described in the literature or else can be prepared according to methods which are described therein or which are known to a person skilled in the art.

The following examples describe the preparation of some compounds in accordance with the invention. These examples are not limiting and serve only to illustrate the present invention. The numbers of the compounds exemplified refer to those given in the table below, in which the chemical structures and the physical properties of some compounds according to the invention are illustrated.

### EXAMPLE 1

### Compound No. 1

### {3-[2-(Piperidin-1-ylcarbonyl)imidazo[1,2-α]pyridin-6-yl]phenyl}methanol

253 μl of a 2M solution of trimethylaluminium in toluene are added, under argon and at 0° C., to a solution of 40 μl of piperidine in 1 ml of xylene. The ice bath is removed and 100 mg of 6-[3-(hydroxymethyl)phenyl]imidazo[1,2-α]pyridine-2-carboxylic acid (Intermediate 3) are added. The reaction mixture is stirred at 110° C. for 30 hours and then diluted with water, acidified to pH 3 with 1N hydrochloric acid and extracted with ethyl acetate and then dichloromethane. The combined organic phases are dried over magnesium sulphate and concentrated to dryness under reduced pressure. The residue is purified by chromatography on silica, elution being carried out with a gradient of hexane, ethyl acetate and methanol (from 47/50/3 to 0/84/16), to give 72 mg of {3-[2-(piperidin-1-ylcarbonyl)imidazo[1,2-α]pyridin-6-yl]phenyl}methanol in the form of a white solid.

### EXAMPLE 2

### Compound No. 2

### {3-[2-(2,3-Dihydro-4H-1,4-benzoxazin-4-ylcarbonyl)imidazo[1,2-α]pyridin-6-yl]phenyl}methanol

76 mg of piperidine are added to a suspension, placed under argon, of 50 mg of 6-[3-(hydroxymethyl)phenyl]imidazo[1,2-α]pyridine-2-carboxylic acid (Intermediate 3) and 72 mg of 1-(3-dimethylaminopropyl)-3-ethylcarbodiimide hydrochloride in 2 ml of anhydrous pyridine. The reaction mixture is stirred at 50° C. for 48 hours and then concentrated to dryness under reduced pressure. The residue is diluted with 3 ml of chloroform and washed with 1 ml of water. The organic phase is dried over magnesium sulphate and concentrated to dryness under reduced pressure. The solid is titrated with methanol and filtered off and then dried to give 42 mg of {3-[2-(2,3-dihydro-4H-1,4-benzoxazin-4-ylcarbonyl)imidazo[1,2-α]pyridin-6-yl]phenyl}methanol in the form of a white solid.

### EXAMPLE 3

### Compound No. 3

### {3-[2-(Morpholin-4-ylcarbonyl)imidazo[1,2-α]pyridin-6-yl]phenyl}methanol

24.4 μl of morpholine are added to a mixture of 50 mg of 6-[3-(hydroxymethyl)phenyl]imidazo[1,2-α]pyridine-2-carboxylic acid (Intermediate 3), 95.5 μl of diisopropylethylamine, 107 mg of 1-[bis](dimethylamino)methylene]-1H-1,2,3-triazolo[4,5-b]pyridinium 1-oxide hexafluorophosphate (HATU) and 38 mg of 1-hydroxy-7-azabenzotriazole (HOAt) in 1 ml of N,N-dimethylformamide. The reaction mixture is stirred at 25° C. for 16 hours, diluted with 1.6 ml of a saturated aqueous sodium hydrogencarbonate solution and 1 ml of water, and then stirred for 30 minutes. The solid is filtered off and washed with 5 ml of a saturated aqueous sodium hydrogencarbonate solution, then twice with 5 ml of water and twice with 5 ml of hexane, to give 48 mg of {3-[2-(morpholin-4-ylcarbonyl)imidazo[1,2-α]pyridin-6-yl]phenyl}methanol in the form of a beige-pink solid.

### EXAMPLE 4

### Compound No. 4

### 2-(1,2,5,6-tetrahydropyridin-1-yl)carbonyl-N,N-dimethylimidazo[1,2-α]pyridin-6-amine

160 μl of 1,2,5,6-tetrahydropyridine are added to a suspension, placed under argon, of 120 mg of 6-dimethylaminoimidazo[1,2-α]pyridine-2-carboxylic acid (Intermediate 1) and 224 mg of 1-(3-dimethylaminopropyl)-3-ethylcarbodiimide hydrochloride in 2 ml of anhydrous pyridine. The reaction mixture is stirred at 50° C. for 48 hours and then concentrated to dryness under reduced pressure. The residue is purified by chromatography on silica, elution being carried out with a gradient of hexane, ethyl acetate and methanol (from 47/50/3 to 0/84/16), to give 90 mg of 2-(1,2,5,6-tetrahydropyridin-1-yl)carbonyl-N,N-dimethylimidazo[1,2-α]pyridin-6-amine in the form of a white solid.

The intermediates defined below are of use in the preparation of the compounds of the present invention.

### Intermediate 1: Ethyl 6-dimethylaminoimidazo[1,2-α]pyridine-2-carboxylate and 6-dimethylaminoimidazo[1,2-α]pyridine-2-carboxylic acid

1.1: Ethyl 6-dimethylaminoimidazo[1,2-α]pyridine-2-carboxylate

26.2 ml of ethyl bromopyruvate are added to a solution of 19.05 g of 5-dimethylaminopyridine-2-amine (J. Chem. Soc. Perkin 1, 68 (1973)) in 380 ml of DME. The reaction mixture is stirred at 20° C. for 6 hours and then, after addition of 380 ml of ethanol, a reflux for 20 hours and, finally, after cooling, concentrated under reduced pressure. The solid is taken up twice in 350 ml of ethyl ether at reflux and filtered hot, then twice in 350 ml of ethyl acetate at reflux and filtered hot, to give 39.66 g of crude ethyl 6-dimethylaminoimidazo[1,2-α]pyridine-2-carboxylate hydrobromide. This salt is taken up in 800 ml of water and treated with solid sodium carbonate, while stirring vigorously, until a pH of 8-9 is reached. The aqueous phase is extracted three times with 500 ml of dichloromethane and the combined organic phases are dried over magnesium sulphate, filtered and concentrated to dryness. The residue is purified by flash chromatography on a silica column, elution being carried out with mixtures of hexane and ethyl acetate (from 5/1 to 1/1), to give 16.7 g of ethyl 6-dimethylaminoimidazo[1,2-α]pyridine-2-carboxylate in the form of a green oil.

¹H NMR spectrum (d₆-DMSO, δ in ppm): 8.35 (s, 1H), 7.81 (d, J=2.2, 1H), 7.45 (d, J=10, 1H), 7.34 (dd, J=2.4, 10, 1H), 4.27 (q, J=7.1, 2H), 2.84 (s, 6H), 1.31 (t, J=7.1, 3H).

1.2: 6-Dimethylaminoimidazo[1,2-α]pyridine-2-carboxylic acid

107 ml of a 2N aqueous lithium hydroxide solution are added at 0° C. to a suspension of 16.7 g of ethyl 6-dimethylaminoimidazo[1,2-α]pyridine-2-carboxylate in a mixture of 220 ml of tetrahydrofuran and 9.5 ml of methanol. The reaction mixture is subsequently reheated to 20° C. and stirred for 4 hours. 2N hydrochloric acid is added dropwise to the reaction mixture, cooled to 0° C., until a pH of 4-5 is reached. The precipitate is filtered off and washed twice with 50 ml of ethyl ether to give 14.8 g of 6-dimethylaminoimidazo[1,2-α]pyridine-2-carboxylic acid in the form of a yellow solid.

¹H NMR spectrum (d₆-DMSO, δ in ppm): 8.67 (s, 1H), 8.18 (d, J=2, 1H), 7.88 (dd, J=2.4, 10, 1H), 7.75 (d, J=10, 1H), 2.96 (s, 6H), (1 acidic H not very visible).

### Intermediate 2: Ethyl 6-methoxy-5-methylimidazo[1,2-α]pyridine-2-carboxylate and 6-methoxy-5-methylimidazo[1,2-α]pyridine-2-carboxylic acid

2.1: Ethyl 6-methoxy-5-methylimidazo[1,2-α]pyridine-2-carboxylate

This product is prepared analogously to ethyl 6-dimethylaminoimidazo[1,2-α]pyridine-2-carboxylate by using 5-methoxy-6-methylpyridine-2-amine in place of 5-dimethylaminopyridine-2-amine.

¹H NMR spectrum (d₆-DMSO, δ in ppm): 1.37 (d, J=6.8 Hz, 3H), 2.62 (s, 3H), 3.92 (s, 3H), 4.42 (q, J=6.8 Hz, 2H), 7.70 (d, J=9.8 Hz, 1H), 7.87 (broad d, J=9.8 Hz, 1H), 8.73 (broad s, 1H).

2.2 6-Methoxy-5-methylimidazo[1,2-α]pyridine-2-carboxylic acid

This product is prepared by saponifying ethyl 6-methoxy-5-methylimidazo[1,2-α]pyridine-2-carboxylate under conditions analogous to those described for the preparation of 6-dimethylaminoimidazo[1,2-α]pyridine-2-carboxylic acid.

¹H NMR spectrum (d₆-DMSO, δ in ppm): 2.52 (s, 3H), 3.85 (s, 3H), 7.47 (d, J=9.8 Hz, 1H), 7.54 (d, J=9.8 Hz, 1H), 8.29 (s, 1H).

### Intermediate 3: Ethyl 6-[3-(Hydroxymethyl)phenyl]imidazo[1,2-α]pyridine-2-carboxylate and 6-[3-(hydroxymethyl)phenyl]imidazo[1,2-α]pyridine-2-carboxylic acid

3.1: Ethyl 6-[3-(Hydroxymethyl)phenyl]imidazo[1,2-α]pyridine-2-carboxylate

475 ml of a mixture of toluene and water (5/1), degassed beforehand, are added to a mixture, under an argon atmosphere, of 25 g of ethyl 6-bromoimidazo[1,2-α]pyridine-2-carboxylate, 13 g of 3-(hydroxymethyl)phenylboronic acid, 5 g of 2-(dicyclohexylphosphino)biphenyl, 1.6 g of palladium acetate and 19 g of potassium phosphate. The reaction mixture is stirred at 80° C. for 16 h, then cooled and diluted with water. After extracting with 2 times 200 ml of dichloromethane, the combined organic phases are dried over sodium sulphate, filtered and concentrated to dryness. The residue is purified by flash chromatography on a silica column, elution being carried out with mixtures of ethyl acetate and methanol (from 100/0 to 96/4), to give 16.1 g of ethyl 6-[3-(hydroxymethyl)phenyl]imidazo[1,2-α]pyridine-2-carboxylate in the form of a light yellow solid.

¹H NMR spectrum (d₆-DMSO, δ in ppm): 8.93 (s, 1H), 8.55 (s, 1H), 7.71-7.66 (m, 3H), 7.57 (d, J=7.7, 1H), 7.48 (t, J=7.6, 1H), 7.39 (d, J=7.5, 1H), 5.29 (t, J=5.7, 1H), 4.61 (d, 5.66, 2H), 4.32 (q, J=7.1, 2H), 1.34 (t, J=7.1, 3H).

3.2: 6-[3-(Hydroxymethyl)phenyl]imidazo[1,2-α]pyridine-2-carboxylic acid

90 ml of a 2N aqueous lithium hydroxide solution are added to a suspension of 17.9 g of ethyl 6-[3-(hydroxymethyl)phenyl]imidazo[1,2-α]pyridine-2-carboxylate in a mixture of 180 ml of tetrahydrofuran and 9 ml of methanol. The reaction mixture is subsequently stirred at 20° C. for 30 minutes. 2N hydrochloric acid is added dropwise to the reaction mixture, cooled to 0° C., until a pH of 4-5 is reached. The precipitate is filtered off and washed twice with 50 ml of ethyl ether to give 15.3 g of 6-[3-(hydroxymethyl)phenyl]imidazo[1,2-α]pyridine-2-carboxylic acid in the form of a white solid.

¹H NMR spectrum (d₆-DMSO, δ in ppm): 8.97 (s, 1H), 8.52 (s, 1H), 7.77-7.67 (m, 3H), 7.57 (d, J=7.7, 1H), 7.48 (t, J=7.6, 1H), 7.39 (d, J=7.5, 1H), 5.7-4.8 (broad s, 1H), 4.60 (s, 2H), (1 acidic H not very visible).

### Intermediate 4: 6-(6-{[(1,1-dimethylethoxy)carbonyl]amino}pyridin-2-yl)imidazo-[1,2-α]pyridine-2-carboxylic acid

4.1: Ethyl 6-(6-aminopyridin-2-yl)imidazo[1,2-α]pyridine-2-carboxylate

350 mg of 2-amino-6-bromopyridine, 750 mg of 2-ethoxycarbonylimidazo[1,2-α]pyridine-6-boronic acid and 57 mg of [1,1′-bis(diphenylphosphino)ferrocene]dichloropalladium are degassed under vacuum and then suspended, under argon, in 20 ml of degassed dioxane. After addition of 2 ml of 2N aqueous sodium carbonate solution, the mixture is degassed under vacuum, then placed under argon and heated at 90° C. for 5 hours, then cooled, diluted and stirred in a mixture of 50 ml of saturated sodium bicarbonate solution and 50 ml of dichloromethane. The organic phase is dried over sodium sulphate, filtered and concentrated to dryness under reduced pressure. The residue is chromatographed on silica, elution being carried out with a mixture of ethyl acetate and hexane. The fractions comprising the expected product are combined and concentrated to dryness under reduced pressure to give 446 mg of ethyl 6-(6-aminopyridin-2-yl)imidazo[1,2-α]pyridine-2-carboxylate.

¹H NMR spectrum (d₆-DMSO, δ in ppm): 9.13 (dd, J=1.0, 1.6 1H), 8.61 (d, J=0.7, 1H), 7.94 (dd, J=1.8, 9.6, 1H), 7.65 (d, J=9.6, 1H), 7.50 (t, J=8.1, 1H), 7.07 (d, J=7.0, 1H), 6.48 (dd, J=0.3, 8.1, 1H), 6.08 (broad s, 2H), 4.33 (q, J=7.1, 2H), 1.33 (t, J=7.1, 3H).

Mass spectrum (APCI): m/z=283 [M+H]⁺.

4.2: Ethyl 6-(6-{[(1,1-dimethylethoxy)carbonyl]amino}pyridin-2-yl)imidazo[1,2-α]pyridine-2-carboxylate and ethyl 6-(6-{bis[(1,1-dimethylethoxy)carbonyl]amino}pyridin-2-yl)-imidazo[1,2-α]pyridine-2-carboxylate

1.14 ml of di(tert-butyl) dicarbonate are added to a suspension of 700 mg of ethyl 6-(6-aminopyridin-2-yl)imidazo[1,2-α]pyridine-2-carboxylate and 25 mg of 4-dimethylaminopyridine in 5 ml of acetonitrile. The mixture is stirred at 25° C. for 16 hours and then concentrated. The residue is chromatographed on silica, elution being carried out with a gradient of ethyl acetate and hexane (from 50/50 to 100/0), to give 370 mg of ethyl 6-(6-{bis[(1,1-dimethylethoxy)carbonyl]amino}pyridin-2-yl)imidazo[1,2-α]pyridine-2-carboxylate

¹H NMR spectrum (d₆-DMSO, δ in ppm): 9.23 (s, 1H), 8.65 (s, 1H), 8.06-7.98 (m, 2H), 7.95 (d, J=7.7, 1H), 7.76 (d, J=9.6, 1H), 7.43 (d, J=7.8, 1H), 4.33 (q, J=7.0, 2H), 1.43 (s, 18H), 1.34 (t, J=7.1, 3H).

Mass spectrum (APCI): m/z=483 [M+H]⁺,

and 163 mg of ethyl 6-(6-{[(1,1-dimethylethoxy)carbonyl]amino}pyridin-2-yl)imidazo[1,2-α]pyridine-2-carboxylate,

¹H NMR spectrum (d₆-DMSO, δ in ppm): 9.28 (s, 1H), 8.50 (s, 1H), 8.04-8.00 (m, 2H), 7.95 (d, J=7.8, 1H), 7.70 (d, J=9.6, 1H), 7.38 (d, J=7.9, 1H), 4.31 (q, J=7.0, 2H), 1.39 (s, 9H), 1.33 (t, J=7.1, 3H).

Mass spectrum (APCI): m/z=383 [M+1-1]⁺.

4.3: 6-(6-{[(1,1-Dimethylethoxy)carbonyl]amino}pyridin-2-yl)imidazo[1,2-α]pyridine-2-carboxylic acid

0.9 ml of a 2M aqueous lithium hydroxide solution is added to a solution of 292 mg of ethyl 6-(6-{bis[(1,1-dimethylethoxy)carbonyl]amino}pyridin-2-yl)imidazo[1,2-α]pyridine-2-carboxylate in 4.73 ml of a 50:1 mixture of tetrahydrofuran and methanol. The reaction mixture is stirred at 25° C. for 7 hours and then treated dropwise at 0° C. with 2N hydrochloric acid until a pH of 3 is reached. The precipitate formed after 20 minutes is filtered off, washed with water (20 ml) and diethyl ether (20 ml) and then dried under reduced pressure to give 195 mg of 6-(6-{[(1,1-dimethylethoxy)carbonyl]amino}pyridin-2-yl)imidazo[1,2-α]pyridine-2-carboxylic acid in the form of a beige solid.

¹H NMR spectrum (d₆-DMSO, δ in ppm): 13.5-12.0 (br, 1H), 9.80 (s, 1H), 9.24 (s, 1H), 8.51 (s, 1H), 8.03 (dd, J=1.5, 9.6 1H), 7.88 (app, t, J=8.0, 7.8, 1H), 7.77 (d, J=8.2, 1H), 7.73 (d, J=9.6, 1H), 7.62 (d, J=7.5, 1H), 1.50 (s, 9H)

### Intermediate 5: 6-(Pyridin-2-yl)imidazo[1,2-α]pyridine-2-carboxylic acid

5.1: Ethyl 6-(Pyridin-2-yl)imidazo[1,2-α]pyridine-2-carboxylate

A mixture of 3.18 g of cesium carbonate, 25 ml of dioxane, 9.3 ml of water, 500 mg of 2-iodopyridine, 89 mg of [1,1′-bis(diphenylphosphino)ferrocene]dichloropalladium and 848 mg of ethyl 6-(4,4,5,5-tetramethyl-1,3,2-dioxaborolan-2-yl)imidazo[1,2-α]pyridine-2-carboxylate hydrobromide (1:1) is heated at 110° C. for 2 hours, then partially concentrated and diluted with dichloromethane and filtered. The organic phase is washed with water and dried over magnesium sulphate, filtered and concentrated to dryness under reduced pressure. The residue is chromatographed on a silica cartridge, elution being carried out with a mixture of dichloromethane and cyclohexane (80/20). The fractions comprising the expected product are combined and concentrated to dryness under reduced pressure to give 317 mg of ethyl 6-(pyridin-2-yl)imidazo[1,2-α]pyridine-2-carboxylate in the form of a brown oil.

¹H NMR spectrum (d₆-DMSO, δ in ppm): 1.34 (t, J=7.0 Hz, 3H), 4.33 (q, J=7.0 Hz, 2H), 7.42 (ddd, J=7.5, 5.5, 2.0 Hz, 1H), 7.73 (d, J=9.3 Hz, 1H), 7.85-8.02 (m, 2H), 8.07 (dd, J=9.3, 2.0 Hz, 1H), 8.64 (s, 1H), 8.70 (broad d, J=5.5 Hz, 1H), 9.36 (broad s, 1H).

Mass spectrum (LC-MS-DAD-ELSD): m/z 268 [M+1-1]⁺.

5.2: 6-(Pyridin-2-yl)imidazo[1,2-α]pyridine-2-carboxylic acid

317 mg of ethyl 6-(pyridin-2-yl)imidazo[1,2-α]pyridine-2-carboxylate are saponified under conditions analogous to those described for the preparation of Intermediate 4 (stage 4.3) to give 280 mg of 6-(pyridin-2-yl)imidazo[1,2-α]pyridine-2-carboxylic acid in the form of a pasty pink solid.

¹H NMR spectrum (d₆-DMSO, δ in ppm): 7.47 (m, 1H), 7.83 (d, J=9.8 Hz, 1H), 7.99 (dt, J=8.5, 2.0 Hz, 1H), 8.06 (d, J=8.5 Hz, 1H), 8.31 (broad d, J=9.8 Hz, 1H), 8.73 (m, 2H), 9.52 (broad s, 1H).

Mass spectrum (LC-MS-DAD-ELSD): m/z 240 [M+H]⁺.

### Intermediate 6: Ethyl 6-(1-triphenylmethyl-1H-imidazol-4-yl)imidazo[1,2-α]pyridine-2-carboxylate and 6-(1-triphenylmethyl-1H-imidazol-4-yl)imidazo[1,2-α]pyridine-2-carboxylic acid

6.1: Ethyl 6-(1-triphenylmethyl-1H-imidazol-4-yl)imidazo[1,2-α]pyridine-2-carboxylate

873 mg of 4-iodo-1-(triphenylmethyl)imidazole, 750 mg of 2-ethoxycarbonyl-imidazo[1,2-α]pyridine-6-boronic acid, 23 mg of palladium acetate and 70 mg of (2-biphenyl)-dicyclohexylphosphine are degassed under vacuum and then suspended, under argon, in a degassed mixture of 15 ml of toluene, 5 ml of water and 5 ml of N-methylpyrrolidone. After addition of 950 mg of potassium phosphate, the mixture is degassed under vacuum, then placed under argon and heated at 100° C. for 15 minutes under microwave irradiation, and then cooled, diluted and stirred in a mixture of 50 ml of saturated sodium bicarbonate solution and 50 ml of dichloromethane. The organic phase is dried over sodium sulphate, filtered and concentrated to dryness under reduced pressure. The residue is chromatographed on silica, elution being carried out with a mixture of ethyl acetate and hexane. The fractions comprising the expected product are combined and concentrated to dryness under reduced pressure to give 508 mg of ethyl 6-(1-triphenylmethyl-1H-imidazol-4-yl)imidazo[1,2-α]pyridine-2-carboxylate.

¹H NMR spectrum (d₆-DMSO, δ in ppm): 8.97 (s, 1H), 8.54 (s, 1H), 7.76-7.72 (m, 1H), 7.56-7.52 (m, 3H), 7.47-7.37 (m, 9H), 7.20-7.17 (m, 6H), 4.31-4.27 (m, 2H), 1.34-1.20 (m, 3H).

Mass spectrum (APCI): m/z=499 [M+H]⁺.

6.2: 6-(1-Triphenylmethyl-1H-imidazol-4-yl)imidazo[1,2-α]pyridine-2-carboxylic acid

500 mg of ethyl 6-(1-triphenylmethyl-1H-imidazol-4-yl)imidazo[1,2-α]pyridine-2-carboxylate are saponified under conditions analogous to those described for the preparation of Intermediate 4 (stage 4.3) to give 346 mg of 6-(1-triphenylmethyl-1H-imidazol-4-yl)imidazo[1,2-α]pyridine-2-carboxylic acid.

¹H NMR spectrum (d₆-DMSO, δ in ppm): 9.01 (s, 1H), 8.51 (s, 1H), 7.83 (d, J=9.5, 1H), 7.59-7.56 (m, 3H), 7.47-7.37 (m, 9H), 7.20-7.17 (m, 6H). No exchangeable proton is observed

Mass spectrum (APCI): m/z=471 [M+H]⁺.

### Intermediate 7: 6-(1H-pyrrol-3-yl)imidazo[1,2-α]pyridine-2-carboxylic acid

7.1 Ethyl 6-[1-(triisopropylsilyl)-1H-pyrrol-3-yl]imidazo[1,2-α]pyridine-2-carboxylate

100 mg of ethyl 6-iodoimidazo[1,2-α]pyridine-2-carboxylate, 135 mg of 1-(triisopropylsilyl)pyrrole-3-boronic acid and 18 mg of tetrakis(triphenylphosphine)palladium(0) are degassed under vacuum and then suspended, under argon, in a degassed mixture of 1.5 ml of 1,2-dimethoxyethane, 1.5 ml of ethanol and 316 μl of 2N aqueous sodium carbonate solution. The reaction mixture is heated at reflux for 4 hours, then cooled and diluted and stirred with a mixture of 5 ml of a semisaturated aqueous sodium bicarbonate solution and 5 ml of dichloromethane. The organic phase is dried over sodium sulphate, filtered and concentrated to dryness under reduced pressure. The residue is chromatographed on silica, elution being carried out with a mixture of ethyl acetate and hexane (50/50). The fractions comprising the expected product are combined and concentrated to dryness under reduced pressure to give 121 mg of ethyl 6-[1-(triisopropylsilyl)-1H-pyrrol-3-yl]imidazo[1,2-α]pyridine-2-carboxylate.

¹H NMR spectrum (d₆-DMSO, δ in ppm): 8.76 (s, 1H), 8.42 (s, 1H), 7.70 (dd, J=1.9, 9.7 1H), 7.59 (d, J=9.7 1H), 7.37 (broad s, 1H), 6.94 (m, 1H), 6.63 (m, 1H), 4.33 (q, J=6.9, 2H), 1.61-1.50 (m, 3H), 1.33 (t, J=6.9, 3H), 1.10-1.03 (m, 18H).

Mass spectrum (APCI): m/z=412 [M+H]⁺.

7.2: 6-(1H-Pyrrol-3-yl)imidazo[1,2-α]pyridine-2-carboxylic acid hydrochloride (1:1)

292 mg of ethyl 6-[1-(triisopropylsilyl)-1H-pyrrol-3-yl]imidazo[1,2-α]pyridine-2-carboxylate are saponified under conditions analogous to those described for the preparation of Intermediate 4 (stage 4.3) to give 140 mg of 6-(1H-pyrrol-3-yl)imidazo[1,2-α]pyridine-2-carboxylic acid hydrochloride (1:1) in the form of a white solid.

¹H NMR spectrum (d₆-DMSO, δ in ppm): 11.07 (broad s, 1H), 8.73 (s, 1H), 8.39 (s, 1H), 7.69 (dd, J=1.3, 9.5, 1H), 7.59 (d, J=9.5, 1H), 7.31 (s, 1H), 6.86 (s, 1H), 6.46 (s, 1H).

Mass spectrum (APCI): m/z=228 [M+H]⁺.

### Intermediate 8: 6-(1H-Pyrazol-3-yl)imidazo[1,2-α]pyridine-2-carboxylic acid

8.1: Ethyl 6-(1H-pyrazol-3-yl)imidazo[1,2-α]pyridine-2-carboxylate

This product is prepared under conditions analogous to those described for the preparation of Intermediate 7 (stage 7.1), 1-(triisopropylsilyl)pyrrole-3-boronic acid being replaced with pyrazole-3-boronic acid.

¹H NMR spectrum (d₄-MeOD, δ in ppm): 8.89 (t, J=1.2, 2.4, 1H), 8.45 (d, J=0.6, 1H), 7.89 (d, J=9.0, 1H), 7.76 (broad s, 1H), 7.67 (d, J=9.5, 1H), 6.77 (d, J=2.4, 1H), 4.42 (q, J=7.1, 2H), 1.43 (t, J=7.1, 3H).

Mass spectrum (APCI): m/z=257 [M+H]⁺.

8.2: 6-(1H-pyrazol-3-yl)imidazo[1,2-α]pyridine-2-carboxylic acid

128 mg of ethyl 6-(1H-pyrazol-3-yl)imidazo[1,2-α]pyridine-2-carboxylate are saponified under conditions analogous to those described for the preparation of Intermediate 4 (stage 4.3) to give 113 mg of 6-(1H-pyrazol-3-yl)imidazo[1,2-α]pyridine-2-carboxylic acid.

¹H NMR spectrum (d₆-DMSO, δ in ppm): 13.50-12.50 (broad s, 1H), 9.03 (s, 1H), 8.40 (s, 1H), 7.83-7.80 (m, 2H), 7.63 (d, J=9.4, 1H), 6.74 (s, 1H).

### Intermediate 9: 6-(furan-2-yl)imidazo[1,2-α]pyridine-2-carboxylic acid

9.1: Ethyl 6-(furan-2-yl)imidazo[1,2-α]pyridine-2-carboxylate

This product is prepared under conditions analogous to those described for the preparation of Intermediate 7 (stage 7.1), 1-(triisopropylsilyl)pyrrole-3-boronic acid being replaced with furan-2-boronic acid.

¹H NMR spectrum (d₆-DMSO, δ in ppm): 8.78 (s, 1H), 8.44 (s, 1H), 7.72 (dd, J=1.8, 9.6, 1H), 7.63-7.60 (m, 2H), 6.89 (d, J=3.4, 1H), 6.57 (dd, J=1.8, 3.4, 1H), 4.42 (q, J=7.1, 2H), 1.42 (t, J=7.1, 3H).

Mass spectrum (APCI): m/z=257 [M+H]⁺.

9.2: 6-(Furan-2-yl)imidazo[1,2-α]pyridine-2-carboxylic acid

384 mg of ethyl 6-(furan-2-yl)imidazo[1,2-α]pyridine-2-carboxylate are saponified under conditions analogous to those described for the preparation of Intermediate 1 (stage 1.3) to give 256 mg of 6-(furan-2-yl)imidazo[1,2-α]pyridine-2-carboxylic acid.

¹H NMR spectrum (d₆-DMSO, δ in ppm): 8.86 (s, 1H), 8.38 (s, 1H), 7.80 (dd, J=1.7, 9.5, 1H), 7.67-7.64 (m, 2H), 6.90 (d, J=3.4, 1H), 6.60 (dd, J=1.8, 3.4, 1H).

### Intermediate 10: 6-(Furan-3-yl)imidazo[1,2-α]pyridine-2-carboxylic acid

10.1: Ethyl 6-(furan-3-yl)imidazo[1,2-α]pyridine-2-carboxylate

This product is prepared under conditions analogous to those described for the preparation of Intermediate 7 (stage 7.1), 1-(triisopropylsilyl)pyrrole-3-boronic acid being replaced with furan-3-boronic acid.

¹H NMR spectrum (d₆-DMSO, δ in ppm): 8.86 (s, 1H), 8.45 (s, 1H), 8.28 (s, 1H), 7.82 (s, 1H), 7.66 (s, 2H), 6.95 (s, 1H), 4.31 (q, J=7.1, 2H), 1.33 (t, J=7.1, 3H).

Mass spectrum (APCI): m/z=257 [M+H]⁺.

10.2: 6-(Furan-3-yl)imidazo[1,2-α]pyridine-2-carboxylic acid

384 mg of ethyl 6-(furan-3-yl)imidazo[1,2-α]pyridine-2-carboxylate are saponified under conditions analogous to those described for the preparation of Intermediate 4 (stage 4.3) to give 287 mg of 6-(furan-3-yl)imidazo[1,2-α]pyridine-2-carboxylic acid.

¹H NMR spectrum (d₆-DMSO, δ in ppm): 8.86 (s, 1H), 8.38 (s, 1H), 8.27 (s, 1H), 7.81 (s, 1H), 7.64 (s, 2H), 6.95 (s, 1H).

Mass spectrum (APCI): m/z=229 [M+H]⁺.

### Intermediate 11: 6-[5-(Hydroxymethyl)furan-2-yl]imidazo[1,2-α]pyridine-2-carboxylic acid

11.1: Ethyl 6-(5-formylfuran-2-yl)imidazo[1,2-α]pyridine-2-carboxylate

2 g of ethyl 6-iodoimidazo[1,2-α]pyridine-2-carboxylate, 1.42 g of 5-formylfuran-2-boronic acid and 231 mg of [1,1′-bis(diphenylphosphino)ferrocene]dichloropalladium are degassed under vacuum and then suspended, under argon, in a degassed mixture of 30 ml of dioxane and 9.4 ml of a 2N aqueous sodium carbonate solution. The reaction mixture is heated at 90° C. for 5 hours, then stirred at 20° C. for 16 hours and concentrated to dryness. The residue is chromatographed on silica, elution being carried out with a mixture of ethyl acetate and hexane (90/10), with ethyl acetate and then with a mixture (99/1) of ethyl acetate and methanol. The fractions comprising the expected product are combined and concentrated to dryness under reduced pressure to give 884 mg of ethyl 6-(5-formylfuran-2-yl)imidazo[1,2-α]pyridine-2-carboxylate.

¹H NMR spectrum (d₆-DMSO, δ in ppm): 9.64 (s, 1H), 9.20 (s, 1H), 8.66 (s, 1H), 7.86-7.74 (m, 2H), 7.72 (d, J=3.8, 1H), 7.37 (d, J=3.8, 1H), 4.33 (q, J=7.0, 2H), 1.33 (t, J=7.1, 3H).

Mass spectrum (APCI): m/z=285 [M+H]⁺.

11.2: Ethyl 6-[5-(hydroxymethyl)furan-2-yl]imidazo[1,2-α]pyridine-2-carboxylate 123 mg of sodium borohydride are added to a suspension of 770 mg of ethyl 6-(5-formylfuran-2-yl)imidazo[1,2-α]pyridine-2-carboxylate in 15 ml of ethanol. The reaction mixture is stirred at 25° C. for 90 minutes, then diluted and stirred with 10 ml of dichloromethane and 3 ml of a semisaturated aqueous sodium carbonate solution. The organic phase is separated, dried over magnesium sulphate, filtered and concentrated under reduced pressure. The residue is chromatographed on silica, elution being carried out with a mixture of dichloromethane and methanol (98/2). The fractions comprising the expected product are combined and concentrated to dryness under reduced pressure. The solid obtained is titrated from 5 ml of dichloromethane, filtered off and dried to give 403 mg of ethyl 6-[5-formylfuran-2-yl]imidazo[1,2-α]pyridine-2-carboxylate in the form of a white solid.

¹H NMR spectrum (d₆-DMSO, δ in ppm): 8.89 (s, 1H), 8.60 (s, 1H), 7.70 (m, 2H), 6.98 (d, J=3.3, 1H), 6.45 (d, J=3.3, 1H), 5.30 (t, J=5.3, 1H), 4.47 (d, J=5.6, 2H), 4.32 (q, J=7.1, 2H), 1.32 (t, J=7.1, 3H).

Mass spectrum (APCI): m/z=287 [M+H]⁺.

11.3: 6-[5-(Hydroxymethyl)furan-2-yl]imidazo[1,2-α]pyridine-2-carboxylic acid 400 mg of ethyl 6-[5-(hydroxymethyl)furan-2-yl]imidazo[1,2-α]pyridine-2-carboxylate are saponified under conditions analogous to those described for the preparation of Intermediate 4 (stage 4.3) to give 346 mg of 6-[5-(hydroxymethyl)furan-2-yl]imidazo[1,2-α]pyridine-2-carboxylic acid in the form of a white solid.

¹H NMR spectrum (d₆-DMSO, δ in ppm): 9.06 (s, 1H), 8.73 (s, 1H), 8.03 (d, J=9.5, 1H), 7.82 (d, J=9.5, 1H), 7.09 (d, J=3.3, 1H), 6.49 (d, J=3.2, 1H), 4.49 (s, 2H).

Mass spectrum (APCI): m/z=259 [M+H]⁺.

### Intermediate 12: 6-(Oxazol-2-yl)imidazo[1,2-α]pyridine-2-carboxylic acid

12.1: Ethyl 6-(oxazol-2-yl)imidazo[1,2-α]pyridine-2-carboxylate

1 g of ethyl 6-iodoimidazo[1,2-α]pyridine-2-carboxylate, 350 mg of tetrakis(triphenyl-phosphine)palladium(0) and 360 mg of lithium chloride are degassed under vacuum and then suspended, under argon, in 15 ml of degassed dioxane. After addition of 5 g of 2-[tri(n-butyl)stannyl]oxazole, the reaction mixture is heated at 90° C. for 3.5 hours, then cooled and diluted and stirred with a mixture of 100 ml of 1M aqueous potassium fluoride solution and 200 ml of ethyl acetate. The aqueous phase is extracted with 200 ml of ethyl acetate and the combined organic phases are washed with aqueous sodium chloride solution and dried over sodium sulphate, filtered and concentrated to dryness under reduced pressure. The residue is chromatographed on silica, elution being carried out with a gradient of ethyl acetate and hexane (from 80/20 to 100/0). The fractions comprising the expected product are combined and concentrated to dryness under reduced pressure to give 530 mg of ethyl 6-(oxazol-2-yl)imidazo[1,2-α]pyridine-2-carboxylate in the form of a yellow powder.

¹H NMR spectrum (d₆-DMSO, δ in ppm): 9.30 (d, J=0.8, 1H), 8.68 (s, 1H), 8.30 (s, 1H), 7.85 (dd, J=1.7, 9.5, 1H), 7.79 (d, J=9.5, 1H), 7.44 (d, J=0.6, 1H), 4.33 (q, J=7.0, 2H), 1.33 (t, J=7.1, 3H).

Mass spectrum (APCI): m/z=258 [M+H]⁺.

12.2: 6-(Oxazol-2-yl)imidazo[1,2-α]pyridine-2-carboxylic acid

512 mg of ethyl 6-(oxazol-2-yl)imidazo[1,2-α]pyridine-2-carboxylate are saponified under conditions analogous to those described for the preparation of Intermediate 4 (stage 4.3) to give 365 mg of 6-(oxazol-2-yl)imidazo[1,2-α]pyridine-2-carboxylic acid in the form of a white solid.

¹H NMR spectrum (d₆-DMSO, δ in ppm): 9.41 (s, 1H), 8.73 (s, 1H), 8.34 (s, 1H), 8.05 (dd, J=1.5, 9.5, 1H), 7.86 (d, J=9.5, 1H), 7.48 (s, 1H).

### Intermediate 13: 6-(1H-1,2,4-Triazol-3-yl)imidazo[1,2-α]pyridine-2-carboxylic acid

13.1: Ethyl 6-[ethoxy(imino)methyl]imidazo[1,2-α]pyridine-2-carboxylate

470 mg of sodium ethanethiolate are added to a solution, cooled to 0° C., of 1 g of ethyl 6-cyanoimidazo[1,2-α]pyridine-2-carboxylate (J. Med. Chem. (1998), 41(22), 4317) in a mixture of 15 ml of ethanol and 10 ml of dichloromethane. The reaction mixture is stirred at 25° C. for 5 hours and filtered, and the filtrate is evaporated to dryness. The residue is chromatographed on silica, elution being carried out with a mixture of dichloromethane and methanol (98/2), to give 625 mg of ethyl 6-[ethoxy(imino)methyl]imidazo[1,2-α]pyridine-2-carboxylate in the form of a pale yellow solid.

¹H NMR spectrum (d₆-DMSO, δ in ppm): 9.17 (s, 1H), 9.04 (s, 1H), 8.64 (s, 1H), 7.84 (m, 1H), 7.68 (m, 1H), 4.33 (q, J=7.1, 4H), 1.34 (t, J=7.2, 6H).

Mass spectrum (APCI): m/z=262 [M+H]⁺.

13.2: Ethyl 6-[hydrazino(imino)methyl]imidazo[1,2-α]pyridine-2-carboxylate

0.2 ml of hydrazine hydrate is added dropwise at 0-5° C. to a solution of 625 mg of ethyl 6-[ethoxy(imino)methyl]imidazo[1,2-α]pyridine-2-carboxylate in 12 ml of ethanol. The reaction mixture is stirred for 2 hours, 73 μl of hydrazine hydrate are then added and the mixture is stirred for a further 2 hours while allowing the temperature to rise to 25° C. The reaction mixture is concentrated to dryness under reduced pressure and the residue dried to give 600 mg of ethyl 6-[hydrazino(imino)methyl]imidazo[1,2-α]pyridine-2-carboxylate, which is used without further purification in the continuation of the synthesis.

¹H NMR spectrum (d₆-DMSO, δ in ppm): 8.77 (broad s, 1H), 8.49 (s, 1H), 7.70 (m, 1H), 7.53 (d, J=9.6, 1H), 5.67 (s, 2H), 5.15 (broad s, 2H), 4.33 (q, J=7.1, 2H), 1.32 (t, J=7.1, 3H).

Mass spectrum (APCI): m/z=248 [M+H]⁺.

13.3: Ethyl 6-(1H-1,2,4-triazol-3-3-yl)imidazo[1,2-α]pyridine-2-carboxylate

A suspension of 580 mg of ethyl 6-[hydrazino(imino)methyl]imidazo[1,2-α]pyridine-2-carboxylate in 6 ml of formic acid is heated at 85° C. for 20 hours. The reaction mixture is concentrated to less than 20% of its initial volume and diluted with 20 ml of water. The solid sodium carbonate is added at 0-5° C. until a pH of 8-9 is reached. The precipitate is filtered off and then purified by chromatography on silica, elution being carried out with a mixture of dichloromethane and methanol (98/2), to give 320 mg of ethyl 6-(1H-1,2,4-triazol-3-yl)imidazo[1,2-α]pyridine-2-carboxylate.

¹H NMR spectrum (d₆-DMSO, δ in ppm): 14.5-14.0 (broad s, 1H), 9.25 (s, 1H), 8.69 (s, 1H), 8.63 (broad s, 1H), 7.94 (dd, J=9.5, 1.5, 1H), 7.73 (d, J=9.5, 1H), 4.33 (q, J=7.0, 2H), 1.33 (t, J=7.0, 3H)

Mass spectrum (APCI): m/z=258 [M+H]⁺.

13.4: 6-(1H-1,2,4-Triazol-3-yl)imidazo[1,2-α]pyridine-2-carboxylic acid

320 mg of ethyl 6-(1H-1,2,4-triazol-3-yl)imidazo[1,2-α]pyridine-2-carboxylate are saponified under conditions analogous to those described for the preparation of Intermediate 4 (stage 4.3) to give 238 mg of 6-(1H-1,2,4-triazol-3-yl)imidazo[1,2-α]pyridine-2-carboxylic acid in the form of an off-white solid.

¹H NMR spectrum (d₆-DMSO, δ in ppm): 14.5-14.2 (broad s, 1H), 9.26 (s, 1H), 8.66-8.62 (m, 2H), 7.91 (d, J=9.1, 1H), 7.73 (d, J=9.6, 1H).

### Intermediate 14: 6-(1H-1,2,3-Triazol-4-yl)imidazo[1,2-α]pyridine-2-carboxylic acid

14.1: Ethyl 6-[(trimethylsilyl)ethynyl]imidazo[1,2-α]pyridine-2-carboxylate

A mixture of 4 g of ethyl 6-iodoimidazo[1,2-α]pyridine-2-carboxylate, 2.63 ml of ethynyltrimethylsilane and 888 mg of dichlorobis(triphenylphosphine)palladium is degassed under vacuum. 240 mg of degassed N,N-dimethylformamide and 3.52 ml of triethylamine are added. The reaction mixture is degassed under argon, then stirred at 50° C. for 50 hours, then cooled and diluted with 20 ml of water. The precipitate is filtered off and washed with 5 ml of water and then chromatographed on silica, elution being carried out with mixtures of ethyl acetate and hexane (from 50/50 to 90/10). The fractions comprising the expected product are combined and concentrated to dryness under reduced pressure to give 3.6 g of ethyl 6-[(trimethylsilyl)ethynyl]imidazo[1,2-α]pyridine-2-carboxylate in the form of an off-white solid.

¹H NMR spectrum (d₆-DMSO, δ in ppm): 8.61 (s, 1H), 8.22 (s, 1H), 7.36 (d, J=9.5, 1H), 7.07 (dd, J=9.5, 1.7, 1H), 4.07 (q, J=7.1, 2H), 1.08 (t, J=7.1, 3H), 0.01 (s, 9H).

Mass spectrum (APCI): m/z=287 [M+H]⁺.

14.2: Ethyl 6-ethynylimidazo[1,2-α]pyridine-2-carboxylate

1.58 ml of a 1M solution of tetrabutylammonium fluoride in tetrahydrofuran are added dropwise to a solution, cooled to 0° C., of 500 mg of ethyl 6-[(trimethylsilyl)ethynyl]imidazo[1,2-α]pyridine-2-carboxylate in 10 ml of anhydrous tetrahydrofuran. The reaction mixture is stirred for 30 minutes, 5 ml of water are then added and extraction is carried out 3 times with 20 ml of dichloromethane. The product is purified by chromatography on silica, elution being carried out with mixtures of ethyl acetate and hexane (from 1/3 to 1/1). The fractions comprising the expected product are combined and concentrated to dryness under reduced pressure to give 280 mg of ethyl 6-ethynylimidazo[1,2-α]pyridine-2-carboxylate in the form of a yellow solid.

¹H NMR spectrum (d₆-DMSO, δ in ppm): 8.86 (d, J=1.0, 1H), 8.50 (d, J=0.6, 1H), 7.63 (d, J=9.4, 1H), 7.37 (d, J=1.7, 9.4, 1H), 4.32 (m, 3H), 1.32 (t, J=7.1 Hz, 3H).

Mass spectrum (APCI): m/z=215 [M+H]⁺.

14.3: Ethyl 6-(1H-1,2,3-triazol-4-3-yl)imidazo[1,2-α]pyridine-2-carboxylate

9.8 mg of cuprous iodide are added to a solution of 220 mg of ethyl 6-ethynylimidazo[1,2-α]pyridine-2-carboxylate and 0.21 ml of azidotrimethylsilane in 4 ml of a mixture (9/1) of N,N-dimethylformamide and methanol. The reaction mixture is stirred at 100° C. for 2 hours, then cooled and diluted with 4 ml of dichloromethane, then filtered through alumina and concentrated to dryness. The residue is chromatographed on silica, elution being carried out with a mixture of dichloromethane and ethanol (97/3). The fractions comprising the expected product are combined and concentrated to dryness under reduced pressure to give 125 mg of ethyl 6-(1H-1,2,3-triazol-4-yl)imidazo[1,2-α]pyridine-2-carboxylate in the form of an off-white solid.

¹H NMR spectrum (d₆-DMSO, δ in ppm): 15.5-15.0 (broad s, 1H), 9.14 (dd, J=1.1, 1.5, 1H), 8.60 (d, J=0.5, 1H), 8.40 (broad s, 1H), 7.82 (dd, J=1.7, 9.5, 1H), 7.75 (d, J=9.5, 1H), 4.33 (q, J=7.1, 2H), 1.33 (t, J=7.1, 3H).

Mass spectrum (APCI): m/z=258 [M+H]⁺.

14.4: 6-(1H-1,2,3-Triazol-4-3-yl)imidazo[1,2-α]pyridine-2-carboxylic acid

125 mg of ethyl 6-(1H-1,2,3-triazol-4-yl)imidazo[1,2-α]pyridine-2-carboxylate are saponified under conditions analogous to those described for the preparation of Intermediate 4 (stage 4.3) to give 72 mg of 6-(1H-1,2,3-triazol-4-yl)imidazo[1,2-α]pyridine-2-carboxylic acid in the form of a white solid.

¹H NMR spectrum (d₆-DMSO, δ in ppm): 16.0-15.0 (broad s, 1H), 9.23 (s, 1H), 8.62 (s, 1H), 8.46 (broad s, 1H), 7.96 (dd, J=1.4, 9.5, 1H), 7.80 (d, J=9.5, 1H).

The chemical structures of general formula (I) (Table 1) and the spectroscopic characteristics (Table 2) of some examples of compounds according to the invention are illustrated in the following tables.

In Table 1:
the “salt” column, “-” represents a compound in the free base form,
“HCl” represents a compound in the hydrochloride form,
“CF₃COOH” represents a compound in the trifluoroacetate form,
the ratio in brackets is the (base:acid) ratio.

TABLE 1
(I)

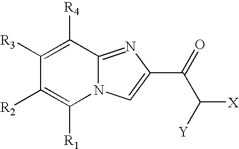

| Ex | R₁ | R₂ | R₃ | R₄ | ~NXY | Salt |
|---|---|---|---|---|---|---|
| 01 | H | 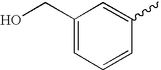 | H | H | 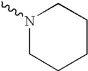 | — |
| 02 | H | 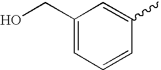 | H | H | 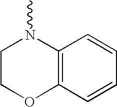 | — |
| 03 | H | 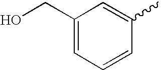 | H | H | 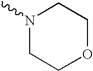 | — |
| 04 | H | ~NMe₂ | H | H | 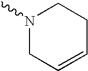 | — |
| 05 | H | 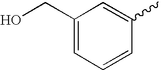 | H | H | 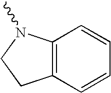 | — |
| 06 | H | 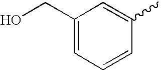 | H | H | 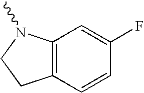 | — |
| 07 | H | 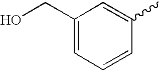 | H | H | 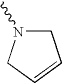 | — |
| 08 | H | 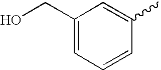 | H | H | 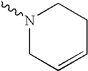 | — |
| 09 | H | 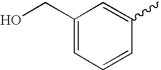 | H | H | 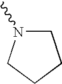 | — |
| 10 | H | 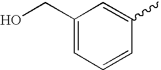 | H | H | 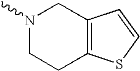 | — |
| 11 | H | ~NMe₂ | H | H | 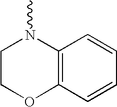 | — |
| 12 | H | 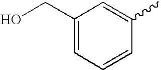 | H | H | 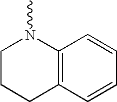 | — |
| 13 | H | 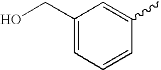 | H | H | 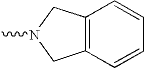 | — |
| 14 | H | ~NMe₂ | H | H | 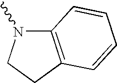 | — |
| 15 | H | ~NMe₂ | H | H | 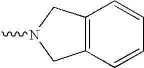 | — |
| 16 | H | ~NMe₂ | H | H | 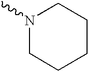 | — |
| 17 | H | ~NMe₂ | H | H | 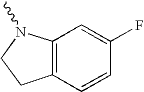 | — |
| 18 | H | ~NMe₂ | H | H | 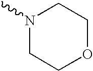 | — |
| 19 | H | ~NMe₂ | H | H | 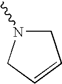 | — |
| 20 | H | 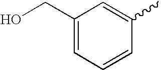 | H | H | 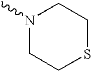 | — |
| 21 | H | ~NMe₂ | H | H | 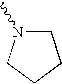 | — |
| 22 | H | ~NMe₂ | H | H | 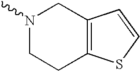 | — |
| 23 | H | ~NMe₂ | H | H | 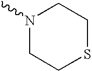 | — |
| 24 | Me | ~OMe | H | H | 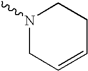 | — |
| 25 | H | 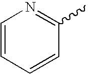 | H | H | 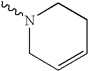 | — |
| 26 | H | 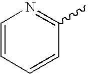 | H | H | 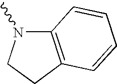 | — |
| 27 | H | 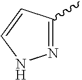 | H | H | 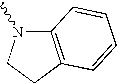 | — |
| 28 | H | 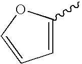 | H | H | 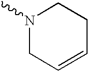 | — |
| 29 | H | 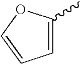 | H | H | 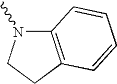 | — |
| 30 | H | 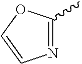 | H | H | 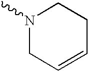 | — |
| 31 | H | 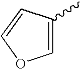 | H | H | 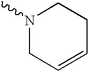 | — |
| 32 | H | 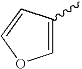 | H | H | 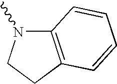 | — |
| 33 | H | 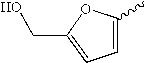 | H | H | 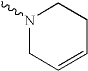 | — |
| 34 | H | 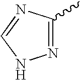 | H | H | 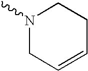 | — |
| 35 | H | 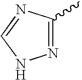 | H | H | 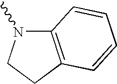 | — |
| 36 | H | 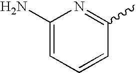 | H | H | 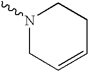 | HCl (1:2) |
| 37 | H | 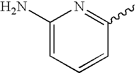 | H | H | 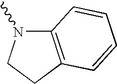 | HCl (1:2) |
| 38 | H | 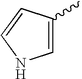 | H | H | 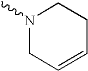 | — |
| 39 | H | 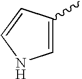 | H | H | 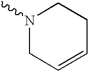 | — |
| 40 | H | 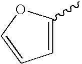 | H | H | 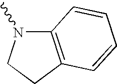 | CF₃COOH (1:1) |
| 41 | H | 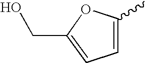 | H | H | 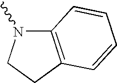 | CF₃COOH (1:1) |
| 42 | H | 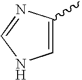 | H | H | 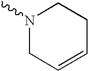 | — |
| 43 | H | 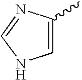 | H | H | 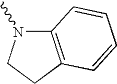 | — |
| 44 | H | 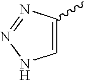 | H | H | 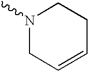 | — |
| 45 | H | 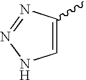 | H | H | 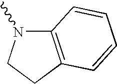 | — |
| 46 | H | 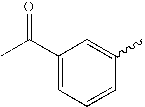 | H | H | 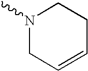 | — |
| 47 | H | 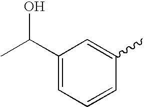 | H | H | 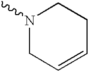 | — |
| 48 | H | 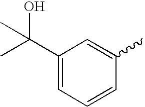 | H | H | 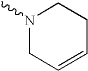 | — |

TABLE 2

| Ex | Characterizations |
|---|---|
| 01 | ¹H NMR spectrum (d₆-DMSO, δ in ppm): 1.48 to 1.70 (m, 6H), 3.62 (broad unresolved m, |
| | 2H), 4.08 (broad unresolved m, 2H), 4.59 (d, J = 5.5 Hz, 2H), 5.27 (t, J = 5.5 Hz, 1H), |
| | 7.37 (broad d, J = 7.5 Hz, 1H), 7.48 (t, J = 7.5 Hz, 1H), 7.58 (broad d, J = 7.5 Hz, 1H), from 7.62 |
| | to 7.73 (m, 3H), 8.30 (s, 1H), 8.91 (t, J = 1.5 Hz, 1H). |
| | Mass spectrum (LC-MS-DAD-ELSD): m/z 336 [M + H]⁺. |
| 02 | ¹H NMR spectrum (d₆-DMSO, δ in ppm): 4.31 (m, 2H), 4.44 (m, 2H), 4.60 (d, J = 5.7 Hz, 2H), |
| | 5.27 (t, J = 5.7, Hz, 1H), 6.85 (m, 1H), 6.92 (dd, J = 8.1, 1.6 Hz, 1H), 7.05 (m, 1H), 7.38 (dt, |
| | J = 7.6, 1.4 Hz, 1H), 7.48 (t, J = 7.6 Hz, 1H), 7.59 (dt, J = 7.6, 1.4 Hz, 1H), 7.65-7.77 (m, 4H), |
| | 8.46 (s, 1H), 8.96 (t, J = 1.6 Hz, 1H). |
| | Mass spectrum (LC-MS-DAD-ELSD): m/z 386 [M + H]⁺. |
| 03 | ¹H NMR spectrum (d₆-DMSO, δ in ppm): 3.67 (broad unresolved m, 6H), 4.27 (broad |
| | unresolved m, 2H), 4.59 (d, J = 5.5 Hz, 2H), 5.28 (t, J = 5.5 Hz, 1H), 7.37 (broad d, J = 7.5 Hz, |
| | 1H), 7.48 (t, J = 7.5 Hz, 1H), 7.59 (broad d, J = 7.5 Hz, 1H), from 7.61 to 7.79 (m, 3H), |
| | 8.38 (s, 1H), 8.93 (t, J = 1.5 Hz, 1H). |
| | Mass spectrum (LC-MS-DAD-ELSD): m/z 338 [M + H]⁺. |
| 04 | ¹H NMR spectrum (d₆-DMSO, δ in ppm): 2.18 (broad m, 2H), 2.84 (s, 6H), 3.76 (broad m, |
| | 1H), 4.00-4.32 (broad m, 2H), 4.71 (broad m, 1H), 5.65-5.92 (broad m, 2H), 7.28 (dd, |
| | J = 9.9, 2.5 Hz, 1H), 7.47 (dt, J = 9.9, 0.8 Hz, 1H), 7.83 (dd, J = 2.5, 0.8 Hz, 1H), 8.15 (d, J = 0.8 Hz, |
| | 1H). |
| | Mass spectrum (LC-MS-DAD-ELSD): m/z 271 [M + H]⁺. |
| 05 | ¹H NMR spectrum (d₆-DMSO, δ in ppm): 3.23 (t, J = 8.4 Hz, 2H), 4.61 (s, 2H), 4.68 (t, J = 8.4 Hz, |
| | 2H), 7.09 (td, J = 7.4, 1.1 Hz, 1H), 7.23 (broad t, J = 7.4 Hz, 1H), 7.32 (dd, J = 7.4, 1.3 Hz, |
| | 1H), 7.39 (dt, J = 7.6, 1.3 Hz, 1H), 7.50 (t, J = 7.6 Hz, 1H), 7.60 (dt, J = 7.6, 1.3 Hz, 1H), 7.70 (t, |
| | J = 1.3 Hz, 1H), 7.78 (d, J = 9.5 Hz, 1H), 7.86 (dd, J = 9.5, 1.6 Hz, 1H), 8.20 (broad unresolved |
| | m, 1H), 8.63 (s, 1H), 9.04 (t, J = 1.6 Hz, 1H). |
| | Mass spectrum (LC-MS-DAD-ELSD): m/z 370 [M + H]⁺. |
| 06 | ¹H NMR spectrum (d₆-DMSO, δ in ppm): 3.18 (t, J = 8.4 Hz, 2H), 4.60 (d, J = 5.7 Hz, 2H), |
| | 4.80 (t, J = 8.4 Hz, 2H), 5.28 (t, J = 5.7 Hz, 1H), 6.88 (td, J = 8.7, 2.6 Hz, 1H), 7.32 (broad dd, J = 8.7, |
| | 5.9 Hz, 1H), 7.37 (dt, J = 7.6, 1.4 Hz, 1H), 7.48 (t, J = 7.6 Hz, 1H), 7.61 (dt, J = 7.6, 1.4 Hz, 1H). |
| | 7.68 (broad s, 1H), 7.71 (dd, J = 9.7, 1.3 Hz, 1H), 7.74 (broad d, J = 9.7 Hz, 1H), 7.97 (broad d, |
| | J = 9.3 Hz, 1H), 8.56 (s, 1H), 8.99 (t, J = 1.3 Hz, 1H). |
| | Mass spectrum (LC-MS-DAD-ELSD): m/z 388 [M + H]⁺. |
| 07 | ¹H NMR spectrum (d₆-DMSO, δ in ppm): 4.34 (m, 2H), 4.59 (d, J = 5.7 Hz, 2H), 4.85 (m, 2H), |
| | 5.27 (t, J = 5.7 Hz, 1H), 5.91-6.03 (m, 2H), 7.37 (dt, J = 7.6, 1.6 Hz, 1H), 7.47 (t, J = 7.6 Hz, |
| | 1H), 7.58 (dt, J = 7.6, 1.6 Hz, 1H), 7.62-7.69 (m, 2H), 7.72 (dt, J = 9.5, 1.3 Hz, 1H), 8.44 (d, |
| | J = 0.8 Hz, 1H), 8.93 (t, J = 1.3 Hz, 1H). |
| | Mass spectrum (LC-MS-DAD-ELSD): m/z 320 [M + H]⁺. |
| 08 | ¹H NMR spectrum (d₆-DMSO, δ in ppm): 2.20 (broad m, 2H), 3.74 (broad m, 1H), |
| | 4.04-4.25 (broad m, 2H), 4.59 (s, 2H), 4.67 (broad m, 1H), 5.20 (very broad unresolved m, 1H), |
| | 5.70-5.92 (m, 2H), 7.36 (dt, J = 7.6, 1.5 Hz, 1H), 7.47 (t, J = 7.6 Hz, 1H), 7.57 (dt, J = 7.6, 1.5 Hz, |
| | 1H), 7.64-7.76 (m, 3H), 8.36 (s, 1H), 8.94 (t, J = 1.5 Hz, 1H). |
| | Mass spectrum (LC-MS-DAD-ELSD): m/z 334 [M + H]⁺. |
| 09 | ¹H NMR spectrum (d₆-DMSO, δ in ppm): 1.78-1.98 (m, 4H), 3.52 (t, J = 6.7 Hz, 2H), 4.04 (t, |
| | J = 6.7 Hz, 2H), 4.59 (d, J = 5.6 Hz, 2H), 5.26 (t, J = 5.6 Hz, 1H), 7.36 (dt, J = 7.6, 1.5 Hz, 1H), |
| | 7.47 (t, J = 7.6 Hz, 1H), 7.58 (dt, J = 7.6, 1.5 Hz, 1H), 7.65 (dd, J = 9.5, 1.7 Hz, 1H), |
| | 7.68 (masked s, 1H), 7.70 (broad d, J = 9.5 Hz, 1H), 8.37 (broad s, 1H), 8.93 (t, J = 1.7 Hz, 1H). |
| | Mass spectrum (LC-MS-DAD-ELSD): m/z 322 [M + H]⁺. |
| 10 | ¹H NMR spectrum (d₆-DMSO, δ in ppm): 2.96 (broad m, 2H), 3.97 (broad m, 1H), |
| | 4.45 (broad m, 1H), 4.59 (d, J = 5.6 Hz, 2H), 4.73 (broad m, 1H), 5.28 (t, J = 5.7 Hz, 1H), 5.29 (broad |
| | m, 1H), 6.76-7.00 (broad m, 1H), 7.37 (m, 2H), 7.47 (t, J = 7.6 Hz, 1H), 7.58 (dt, J = 7.6, 1.6 Hz, |
| | 1H), 7.64-7.78 (m, 3H), 8.38 (s, 1H), 8.95 (t, J = 1.4 Hz, 1H). |
| | Mass spectrum (LC-MS-DAD-ELSD): m/z 390 [M + H]⁺. |
| 11 | ¹H NMR spectrum (d₆-DMSO, δ in ppm): 2.85 (s, 6H), 4.28 (m, 2H), 4.46 (m, 2H), 6.84 (m, |
| | 1H), 6.90 (dd, J = 8.2, 1.6 Hz, 1H), 7.02 (m, 1H), 7.32 (dd, J = 10.0, 2.4 Hz, 1H), 7.49 (dt, |
| | J = 10.0, 0.9 Hz, 1H), 7.70 (broad dd, J = 8.2, 1.6 Hz, 1H), 7.86 (dd, J = 2.4, 0.9 Hz, 1H), 8.27 (d, |
| | J = 0.9 Hz, 1H). |
| | Mass spectrum (LC-MS-DAD-ELSD): m/z 323 [M + H]⁺. |
| 12 | ¹H NMR spectrum (d₆-DMSO, δ in ppm): 1.94 (m, 2H), 2.83 (t, J = 6.8 Hz, 2H), 4.09 (m, 2H), |
| | 4.59 (d, J = 5.7 Hz, 2H), 5.26 (t, J = 5.7 Hz, 1H), 7.02 (m, 2H), 7.20 (m, 1H), 7.27 (m, 1H), |
| | 7.37 (broad d, J = 7.6 Hz, 1H), 7.46 (t, J = 7.6 Hz, 1H), 7.57 (broad d, J = 7.6 Hz, 1H), 7.66 (d, J = 1.6 Hz, |
| | 3H), 8.28 (s, 1H), 8.92 (t, J = 1.6 Hz, 1H). |
| | Mass spectrum (LC-MS-DAD-ELSD): m/z 384 [M + H]⁺. |
| 13 | ¹H NMR spectrum (d₆-DMSO, δ in ppm): 4.60 (d, J = 5.7 Hz, 2H), 4.92 (broad s, 2H), 5.28 (t, |
| | J = 5.7 Hz, 1H), 5.47 (broad s, 2H), 7.28-7.45 (m, 5H), 7.48 (t, J = 7.6 Hz, 1H), 7.60 (broad d, |
| | J = 7.9 Hz, 1H), 7.68 (masked s, 1H), 7.70 (dd, J = 9.5, 1.8 Hz, 1H), 7.77 (broad d, J = 9.5 Hz, |
| | 1H), 8.50 (s, 1H), 8.98 (broad s, 1H). |
| | Mass spectrum (LC-MS-DAD-ELSD): m/z 370 [M + H]⁺. |
| 14 | ¹H NMR spectrum (d₆-DMSO, δ in ppm): 2.86 (s, 6H), 3.17 (t, J = 8.5 Hz, 2H), 4.71 (t, J = 8.5 Hz, |
| | 2H), 7.03 (td, J = 7.6, 1.3 Hz, 1H), 7.19 (broad t, J = 7.6 Hz, 1H), 7.29 (broad d, J = 7.6 Hz, |
| | 1H), 7.31 (dd, J = 9.9, 2.4 Hz, 1H), 7.51 (d, J = 9.9 Hz, 1H), 7.87 (broad d, J = 2.4 Hz, 1H), |
| | 8.17 (broad unresolved m, 1H), 8.33 (s, 1H). |
| | Mass spectrum (LC-MS-DAD-ELSD): m/z 307 [M + H]⁺. |
| 15 | ¹H NMR spectrum (d₆-DMSO, δ in ppm): 2.86 (s, 6H), 4.89 (broad s, 2H), 5.43 (broad s, 2H), |
| | 7.27-7.35 (m, 3H), 7.41 (m, 2H), 7.54 (broad d, J = 9.9 Hz, 1H), 7.88 (broad d, J = 2.5 Hz, |
| | 1H), 8.30 (s, 1H). |
| | Mass spectrum (LC-MS-DAD-ELSD): m/z 307 [M + H]⁺. |
| 16 | ¹H NMR spectrum (d₆-DMSO, δ in ppm): 1.44-1.70 (m, 6H), 2.83 (s, 6H), 3.58 (broad |
| | unresolved m, 2H), 4.12 (broad unresolved m, 2H), 7.26 (dd, J = 9.9, 2.4 Hz, 1H), 7.46 (dt, |
| | J = 9.9, 1.0 Hz, 1H), 7.83 (dd, J = 2.4, 1.0 Hz, 1H), 8.10 (d, J = 1.0 Hz, 1H). |
| | Mass spectrum (LC-MS-DAD-ELSD): m/z 273 [M + H]⁺. |
| 17 | ¹H NMR spectrum (d₆-DMSO, δ in ppm): 2.86 (s, 6H), 3.15 (t, J = 8.5 Hz, 2H), 4.78 (t, J = 8.5 Hz, |
| | 2H), 6.85 (ddd, J = 9.2, 8.2, 2.3 Hz, 1H), 7.28 (broad dd, J = 8.2, 5.7 Hz, 1H), 7.32 (dd, |
| | J = 9.9, 2.3 Hz, 1H), 7.51 (dt, J = 9.9, 1.0 Hz, 1H), 7.87 (dd, J = 2.3, 1.0 Hz, 1H), 7.95 (broad d, |
| | J = 11.7 Hz, 1H), 8.36 (d, J = 1.0 Hz, 1H). |
| | Mass spectrum (LC-MS-DAD-ELSD): m/z 325 [M + H]⁺. |
| 18 | ¹H NMR spectrum (d₆-DMSO, δ in ppm): 2.83 (s, 6H), 3.64 (broad m, 6H), 4.30 (broad m, |
| | 2H), 7.28 (dd, J = 10.0, 2.4 Hz, 1H), 7.45 (dt, J = 10.0, 0.9 Hz, 1H), 7.84 (dd, J = 2.4, 0.9 Hz, |
| | 1H), 8.18 (d, J = 0.9 Hz, 1H). |
| | Mass spectrum (LC-MS-DAD-ELSD): m/z 275 [M + H]⁺. |
| 19 | ¹H NMR spectrum (d₆-DMSO, δ in ppm): 2.84 (s, 6H), 4.30 (m, 2H), 4.81 (m, 2H), |
| | 5.89-6.01 (m, 2H), 7.28 (dd, J = 9.9, 2.5 Hz, 1H), 7.46 (dd, J = 9.9, 0.9 Hz, 1H), 7.85 (dt, J = 2.5, 0.9 Hz, |
| | 1H), 8.24 (d, J = 0.9 Hz, 1H) |
| | Mass spectrum (LC-MS-DAD-ELSD): m/z 257 [M + H]⁺. |
| 20 | ¹H NMR spectrum (d₆-DMSO, δ in ppm): 2.69 (m, 4H), 3.91 (broad unresolved m, 2H), |
| | 4.42 (broad unresolved m, 2H), 4.59 (d, J = 5.5 Hz, 2H), 5.27 (t, J = 5.5 Hz, 1H), 7.38 (broad d, J = 7.5 Hz, |
| | 1H), 7.47 (t, J = 7.5 Hz, 1H), 7.58 (broad d, J = 7.5 Hz, 1H), 7.53-7.64 (m, 3H), |
| | 8.34 (s, 1H), 8.92 (broad s, 1H). |
| | Mass spectrum (LC-MS-DAD-ELSD): m/z 354 [M + H]⁺. |
| 21 | ¹H NMR spectrum (d₆-DMSO, δ in ppm): 1.69-2.00 (m, 4H), 2.83 (s, 6H), 3.49 (t, J = 6.8 Hz, |
| | 2H), 4.01 (t, J = 6.6 Hz, 2H), 7.26 (dd, J = 9.9, 2.4 Hz, 1H), 7.46 (dt, J = 9.9, 0.9 Hz, 1H), |
| | 7.83 (dd, J = 2.4, 0.9 Hz, 1H), 8.17 (d, J = 0.9 Hz, 1H). |
| | Mass spectrum (LC-MS-DAD-ELSD): m/z 259 [M + H]⁺. |
| 22 | ¹H NMR spectrum (d₆-DMSO, δ in ppm): 2.84 (s, 6H), 2.92 (broad m, 2H), 3.94 (broad m, |
| | 1H), 4.22-4.77 (broad unresolved m, 2H), 5.31 (broad m, 1H), 6.94 (broad unresolved m, |
| | 1H), 7.29 (dd, J = 9.9, 2.3 Hz, 1H), 7.34 (broad d, J = 5.1 Hz, 1H), 7.50 (dt, J = 9.9, 0.9 Hz, 1H), |
| | 7.84 (dd, J = 2.5, 0.9 Hz, 1H), 8.19 (d, J = 0.9 Hz, 1H). |
| | Mass spectrum (LC-MS-DAD-ELSD): m/z 327 [M + H]⁺. |
| 23 | ¹H NMR spectrum (d₆-DMSO, δ in ppm): 2.66 (m, 4H), 2.84 (s, 6H), 3.88 (broad unresolved |
| | m, 2H), 4.45 (broad unresolved m, 2H), 7.28 (dd, J = 9.9, 2.5 Hz, 1H), 7.46 (dt, J = 9.9, 0.9 Hz, |
| | 1H), 7.83 (dd, J = 2.5, 0.9 Hz, 1H), 8.16 (d, J = 0.9 Hz, 1H). |
| | Mass spectrum (LC-MS-DAD-ELSD): m/z 291 [M + H]⁺. |
| 24 | ¹H NMR spectrum (d₆-DMSO, δ in ppm): 2.19 (broad m, 2H), 2.52 (s, 3H), 3.73 (broad m, |
| | 1H), 3.85 (s, 3H), 4.10 (broad m, 1H), 4.19 (broad m, 1H), 4.65 (broad m, 1H), |
| | 5.63-5.93 (broad unresolved m, 2H), 7.45 (d, J = 9.8 Hz, 1H), 7.55 (broad d, J = 9.8 Hz, 1H), 8.13 (broad |
| | s, 1H). |
| | Mass spectrum (LC-MS-DAD-ELSD): m/z 272 [M + H]⁺. |
| 25 | 2.14-2.29 (m, 2H), 3.70-3.80 (m, 1H), 4.06-4.25 (m, 2H), 4.63-4.72 (m, 1H), |
| | 5.71-5.93 (m, 2H), 7.41 (ddd, J = 7.2, 4.8, 1.5 Hz, 1H), 7.72 (d, J = 9.6 Hz, 1H), 7.93-8.07 (m, 3H), |
| | 8.42 (s, 1H), 8.69 (ddd, J = 4.8, 1.5, 0.9 Hz, 1H), 9.35-9.39 (dd, J = 1.5, 0.9 Hz, 1H) |
| | Mass spectrum (LC-MS-ES+/−): m/z 305 [M + H]⁺ |
| 26 | ¹H NMR spectrum (d₆-DMSO, δ in ppm): 3.20 (t, J = 8.4 Hz, 2H), 4.72 (t, J = 8.4 Hz, 2H), |
| | 7.02-7.10 (m, 1H), 7.24 (t, J = 7.5, 1H), 7.29 (d, J = 7.5, 1H), 7.42 (ddd, J = 7.2, 4.9, 1.2 Hz, 1H), |
| | 7.76 (d, J = 9.6 Hz, 1H), 7.91-8.05 (m, 2H), 8.08 (dd, J = 9.6, 1.8 Hz, 1H), 8.16-8.26 (m, |
| | 1H), 8.61 (s, 1H), 8.71 (ddd, J = 4.8, 1.8, 0.9 Hz, 1H), 9.39-9.44 (broad s, 1H) |
| | Mass spectrum (LC-MS-ES+/−): m/z 341 [M + H]⁺ |
| 27 | ¹H NMR spectrum (d₆-DMSO, δ in ppm): 3.20 (t, J = 8.5 Hz, 2H), 4.62-4.82 (t, J = 8.5 Hz, |
| | 2H), 6.76 (d, J = 2.0 Hz, 1H), 7.01-7.13 (t, J = 7.5 Hz, 1H), 7.15-7.26 (t, J = 7.5 Hz, 1H), |
| | 7.30 (d, J = 7.5 Hz, 1H), 7.65-7.76 (d, J = 9.5 Hz, 1H), 7.77-7.94 (m, 2H), 8.20 (broad m, 1H), |
| | 8.52 (s, 1H), 9.07 (s, 1H), 13.03 (s, 1H) |
| | Mass spectrum (LC-MS-ES+/−): m/z 328 [M + H]⁻, m/z 330 [M + H]⁺ |
| 28 | ¹H NMR spectrum (d₆-DMSO, δ in ppm): 2.12-2.32 (m, 2H), 3.64-3.83 (m, 1H), |
| | 4.03-4.28 (m, 2H), 4.58-4.75 (m, 1H), 5.67-5.95 (m, 2H), 6.64 (dd, J = 3.3, 1.8 Hz, 1H), 7.02 (d, |
| | J = 3.3 Hz, 1H), 7.69 (d, J = 1.8 Hz, 2H), 7.80 (d, J = 1.8 Hz, 1H), 8.37 (s, 1H), 8.93 (s, 1H) |
| | Mass spectrum (LC-MS-ES+/−): m/z 294 [M + H]⁺ |
| 29 | ¹H NMR spectrum (d₆-DMSO, δ in ppm): 3.19 (t, J = 8.4 Hz, 2H), 4.71 (t, J = 8.4 Hz, 2H), |
| | 6.66 (dd, J = 3.4, 1.9 Hz, 1H), 7.02-7.09 (m, 2H), 7.21 (broad t, J = 7.6 Hz, 1H), 7.30 (broad d, |
| | J = 7.6 Hz, 1H), 7.72 (m, 2H), 7.82 (dd, J = 1.9, 0.8 Hz, 1H), 8.19 (broad unresolved m, 1H), |
| | 8.56 (s, 1H), 8.98 (broad s, 1H) |
| | Mass spectrum (LC-MS-ES+/−): m/z 330 [M + H]⁺ |
| 30 | ¹H NMR spectrum (d₆-DMSO, δ in ppm): 2.10-2.29 (m, 2H), 3.69-3.78 (m, 1H), |
| | 4.05-4.24 (m, 2H), 4.54-4.72 (m, 1H), 5.67-5.95 (m, 2H), 7.40-7.47 (m, 1H), 7.72-7.78 (d, |
| | J = 9.5 Hz, 1H), 7.80-7.85 (dd, J = 9.5, 1.7 Hz, 1H), 8.29 (m, 1H), 8.48 (s, 1H), 9.32 (broad s, |
| | 1H) |
| | Mass spectrum (LC-MS-ES+/−): m/z 295 [M + H]⁺ |
| 31 | ¹H NMR spectrum (d₆-DMSO, δ in ppm): 2.20 (m, 2H), 3.73 (m, 1H), 4.05-4.26 (m, 2H), |
| | 4.67 (m, 1H), 5.69-5.93 (m, 2H), 6.96 (dd, J = 1.9, 0.9 Hz, 1H), 7.62 (dd, J = 9.5, 1.7 Hz, 1H), |
| | 7.67 (d, J = 9.5 Hz, 1H), 7.80 (t, J = 1.9 Hz, 1H), 8.24 (s, 1H), 8.26 (broad s, 1H), 8.87 (broad s, |
| | 1H) |
| | Mass spectrum (LC-MS-ES+/−): m/z 294 [M + H]⁺ |
| 32 | ¹H NMR spectrum (d₆-DMSO, δ in ppm): 3.19 (t, J = 8.5 Hz, 2H), 4.70 (t, J = 8.5 Hz, 2H), |
| | 6.98 (dd, J = 1.9, 0.9 Hz, 1H), 7.03-7.46 (m, 3H), 7.65 (dd, J = 9.5, 1.7 Hz, 1H), 7.70 (d, J = 9.5 Hz, |
| | 1H), 7.81 (t, J = 1.9 Hz, 1H), 8.18 (broad unresolved m, 1H), 8.28 (broad s, 1H), 8.42 (s, 1H), |
| | 8.92 (broad s, 1H) |
| | Mass spectrum (LC-MS-ES+/−): m/z 330 [M + H]⁺ |
| 33 | ¹H NMR spectrum (d₆-DMSO, δ in ppm): 2.13-2.25 (m, 2H), 3.65-3.79 (m, 1H), |
| | 4.05-4.25 (m, 2H), 4.47 (d, J = 5.7 Hz, 2H), 4.61-4.71 (m, 1H), 5.28 (t, J = 5.7 Hz, 1H), |
| | 5.69-5.93 (m, 2H), 6.45 (d, J = 3.3 Hz, 1H), 6.94 (d, J = 3.3 Hz, 1H), 7.67 (m, 2H), 8.39 (s, 1H), |
| | 8.89 (broad s, 1H) |
| | Mass spectrum (LC-MS-ES+/−): m/z 324 [M + H]⁺ |
| 34 | ¹H NMR spectrum (d₆-DMSO, δ in ppm): 2.10-2.30 (m, 2H), 3.65-3.82 (m, 1H), |
| | 4.05-4.30 (m, 2H), 4.59-4.74 (m, 1H), 5.68-5.95 (m, 2H), 7.72 (d, J = 9.5 Hz, 1H), 7.91 (dd, |
| | J = 9.5, 1.7 Hz, 1H), 8.47 (s, 1H), 8.61 (broad unresolved m, 1H), 9.26 (broad s, 1H), |
| | 14.12-14.43 (broad unresolved m, 1H) |
| | Mass spectrum (LC-MS-ES+/−): m/z 293 [M + H]⁻, m/z 295 [M + H]⁺ |
| 35 | ¹H NMR spectrum (d₆-DMSO, δ in ppm): 3.22 (t, J = 8.5 Hz, 2H), 4.69 (t, J = 8.5 Hz, 2H), |
| | 7.07 (t, J = 7.3 Hz, 1H), 7.22 (t, J = 7.3 Hz, 1H), 7.32 (d, J = 7.3 Hz, 1H), 7.78 (d, J = 9.5 Hz, 1H), |
| | 8.01 (dd, J = 9.5, 1.7 Hz, 1H), 8.19 (broad unresolved m, 1H), 8.63 (broad unresolved m, 1H), |
| | 8.72 (s, 1H), 9.32 (broad s, 1H) |
| | Mass spectrum (LC-MS-ES+/−): m/z 329 [M + H]⁻, m/z 331 [M + H]⁺ |
| 36 | ¹H NMR spectrum (d₆-DMSO, δ in ppm): 2.15-2.33 (m, 2H), 3.70 (masked m, 1H), 4.10 (m, |
| | 2H), 4.57 (m, 1H), 5.63-5.96 (m, 2H), 6.94 (broad d, J = 8.6 Hz, 1H), 7.23 (d, J = 7.5 Hz, 1H), |
| | 7.80-8.01 (m, 3H), 8.05 (broad unresolved m, 3H), 8.48 (s, 1H), 9.29 (broad s, 1H) |
| | Mass spectrum (LC-MS-ES+/−): m/z 320 [M + H]⁺ |
| 37 | ¹H NMR spectrum (d₆-DMSO, δ in ppm): 3.23 (t, J = 8.3 Hz, 2H), 4.66 (t, J = 8.3 Hz, 2H), |
| | 6.96 (d, J = 8.5 Hz, 1H), 7.06-7.13 (m, 1H), 7.20-7.28 (m, 2H), 7.30-7.35 (m, 1H), |
| | 7.84-8.03 (m, 3H), 8.05 (broad unresolved m, 2H), 8.14-8.29 (broad unresolved m, 1H), 8.69 (s, 1H), |
| | 9.34 (broad s, 1H) |
| | Mass spectrum (LC-MS-ES+/−): m/z 356 [M + H]⁺ |
| 38 | ¹H NMR spectrum (d₆-DMSO, δ in ppm): 2.19 (m, 2H), 3.73 (m, 1H), 3.95-4.33 (m, 2H), |
| | 4.69 (m, 1H), 5.64-5.95 (m, 2H), 6.45 (m, 1H), 6.85 (m, 1H), 7.28 (m, 1H), 7.56 (d, J = 9.5 Hz, |
| | 1H), 7.60 (dd, J = 9.5, 1.7 Hz, 1H), 8.21 (s, 1H), 8.70 (broad s, 1H), 11.02 (broad |
| | unresolved m, 1H) |
| | Mass spectrum (LC-MS-ES+/−): m/z 291 [M + H]⁻, m/z 293 [M + H]⁺ |
| 39 | ¹H NMR spectrum (d₆-DMSO, δ in ppm): 3.19 (t, J = 8.5 Hz, 2H), 4.72 (t, J = 8.5 Hz, 2H), |
| | 6.47 (m, 1H), 6.86 (m, 1H), 7.05 (td, J = 7.9, 1.1 Hz, 1H), 7.20 (broad t, J = 7.9 Hz, 1H), |
| | 7.27-7.32 (m, 2H), 7.59 (d, J = 9.5 Hz, 1H), 7.64 (dd, J = 9.5, 1.7 Hz, 1H), 8.19 (broad unresolved m, 1H), |
| | 8.39 (s, 1H), 8.75 (broad s, 1H), 11.03 (broad unresolved m, 1H) |
| | Mass spectrum (LC-MS-ES+/−): m/z 327 [M + H]⁻, m/z 329 [M + H]⁺ |
| 40 | ¹H NMR spectrum (d₆-DMSO, δ in ppm): 3.20 (t, J = 8.3 Hz, 2H), 4.70 (t, J = 8.3 Hz, 2H), |
| | 7.07 (td, J = 7.4, 1.2 Hz, 1H), 7.22 (broad t, J = 7.4 Hz, 1H), 7.31 (broad d, J = 7.4 Hz, 1H), 7.45 (d, |
| | J = 0.9 Hz, 1H), 7.80 (d, J = 9.5 Hz, 1H), 7.86 (dd, J = 9.5, 1.7 Hz, 1H), 8.20 (broad unresolved |
| | m, 1H), 8.30 (d, J = 0.9 Hz, 1H), 8.66 (s, 1H), 9.37 (broad s, 1H) |
| | Mass spectrum (LC-MS-ES+/−): m/z 331 [M + H]⁺ |
| 41 | ¹H NMR spectrum (d₆-DMSO, δ in ppm): 3.20 (t, J = 8.5 Hz, 2H), 4.48 (s, 2H), 4.70 (t, J = 8.5 Hz, |
| | 2H), 5.45 (broad unresolved m, 1H), 6.46 (d, J = 3.4 Hz, 1H), 6.97 (d, J = 3.4 Hz, 1H), |
| | 7.06 (td, J = 7.7, 1.1 Hz, 1H), 7.21 (td, J = 7.6, 1.0 Hz, 1H), 7.30 (dd, J = 7.8, 1.1 Hz, 1H), 7.72 (m, |
| | 2H), 8.19 (broad unresolved m, 1H), 8.59 (s, 1H), 8.94 (broad s, 1H) |
| | Mass spectrum (LC-MS-ES+/−): m/z 360 [M + H]⁺, m/z 404 [M + HCO₂H − H]⁻ |
| 42 | ¹H NMR spectrum (d₆-DMSO, δ in ppm): 2.13-2.29 (m, 2H), 3.66-3.81 (m, 1H), |
| | 4.05-4.29 (m, 2H), 4.62-4.75 (m, 1H), 5.69-5.94 (m, 2H), 7.58-7.79 (m, 4H), 8.35 (s, 1H), |
| | 8.93 (broad s, 1H), 12.26 (broad unresolved m, 1H) |
| | Mass spectrum (LC-MS-ES+/−): m/z 292 [M + H]⁻, m/z 294 [M + H]⁺ |
| 43 | ¹H NMR spectrum (d₆-DMSO, δ in ppm): 3.19 (t, J = 8.5 Hz, 2H), 4.72 (t, J = 8.5 Hz, 2H), |
| | 7.05 (td, J = 7.4, 1.1 Hz, 1H), 7.21 (broad t, J = 7.4 Hz, 1H), 7.30 (broad d, J = 7.4 Hz, 1H), 7.65 (d, |
| | J = 9.5 Hz, 1H), 7.68 (broad s, 1H), 7.74-7.79 (m, 2H), 8.19 (broad unresolved m, 1H), |
| | 8.54 (s, 1H), 8.98 (broad s, 1H), 12.41 (broad unresolved m, 1H) |
| | Mass spectrum (LC-MS-ES+/−): m/z 328 [M + H]⁻, m/z 330 [M + H]⁺ |
| 44 | ¹H NMR spectrum (d₆-DMSO, δ in ppm): 2.08-2.30 (m, 2H), 3.66-3.82 (m, 1H), |
| | 4.05-4.28 (m, 2H), 4.59-4.77 (m, 1H), 5.68-5.97 (m, 2H), 7.72 (d, J = 9.5 Hz, 1H), 7.80 (dd, |
| | J = 9.5, 1.7 Hz, 1H), 8.38 (s, 1H), 8.40 (s, 1H), 9.14 (broad s, 1H), 15.23 (broad unresolved m, |
| | 1H) |
| | Mass spectrum (LC-MS-ES+/−): m/z 293 [M + H]⁻, m/z 295 [M + H]⁺ |
| 45 | ¹H NMR spectrum (d₆-DMSO, δ in ppm): 3.20 (d, J = 8.4 Hz, 2H), 4.71 (t, J = 8.4 Hz, 2H), |
| | 7.06 (t, J = 7.5 Hz, 1H), 7.20 (t, J = 7.5 Hz, 1H), 7.29 (d, J = 7.5 Hz, 1H), 7.76 (d, J = 9.5 Hz, 1H), |
| | 7.83 (broad d, J = 9.5 Hz, 1H), 8.19 (broad unresolved m, 1H), 8.41 (s, 1H), 8.56 (s, 1H), |
| | 9.18 (broad s, 1H), 15.19 (broad unresolved m, 1H) |
| | Mass spectrum (LC-MS-ES+/−): m/z 329 [M + H]⁻, m/z 331 [M + H]⁺ |
| 46 | ¹H NMR spectrum (d₆-DMSO, δ in ppm): 2.23 (m, 2H), 2.68 (s, 3H), 3.75 (m, 1H), |
| | 4.02-4.28 (m, 2H), 4.68 (m, 1H), 5.67-5.98 (m, 2H), 7.68 (t, J = 7.8 Hz, 1H), 7.71-7.79 (m, 2H), |
| | 7.97-8.02 (m, 2H), 8.26 (t, J = 1.8 Hz, 1H), 8.35 (s, 1H), 9.05 (broad s, 1H) |
| | Mass spectrum (LC-MS-ES+/−): m/z 346 [M + H]⁺ |
| 47 | ¹H NMR spectrum (d₆-DMSO, δ in ppm): 1.39 (d, J = 6.7 Hz, 3H), 2.22 (m, 2H), 3.74 (m, 1H), |
| | 4.06-4.26 (m, 2H), 4.68 (m, 1H), 4.81 (m, 1H), 5.23 (d, J = 4.5 Hz, 1H), 5.69-5.92 (m, 2H), |
| | 7.38 (broad d, J = 7.8 Hz, 1H), 7.45 (t, J = 7.8 Hz, 1H), 7.56 (broad d, J = 7.8 Hz, 1H), |
| | 7.63-7.78 (m, 3H), 8.34 (s, 1H), 8.93 (broad s, 1H) |
| | Mass spectrum (LC-MS-ES+/−): m/z 348 [M + H]⁺ |
| 48 | ¹H NMR spectrum (d₆-DMSO, δ in ppm): 1.49 (s, 6H), 2.15-2.29 (m, 2H), 3.74 (m, 1H), |
| | 4.05-4.26 (m, 2H), 4.68 (m, 1H), 5.11 (s, 1H), 5.69-5.95 (m, 2H), 7.43 (t, J = 7.7 Hz, 1H), |
| | 7.49-7.55 (m, 2H), 7.67 (dd, J = 9.5, 1.8 Hz, 1H), 7.72 (broad d, J = 9.5 Hz, 1H), 7.80 (t, J = 1.8 Hz, |
| | 1H), 8.35 (s, 1H), 8.92 (broad s, 1H) |
| | Mass spectrum (LC-MS-ES+/−): m/z 362 [M + H]⁺ |

The compounds according to the invention have formed the subject of pharmacological assays which make it possible to determine their modulatory effect on NOT.

Evaluation of the in vitro Activity on N2A Cells

The activity of the compounds according to the invention was evaluated on a cell line (N2A) endogenously expressing the mouse Nurr1 receptor and stably transfected with the NOT binding response element (NBRE) coupled to the luciferase reporter gene. The EC₅₀ values are between 0.01 and 1000 nM. The assays were carried out according to the procedure described below.

The Neuro-2A cell line comes from a standard commercial source (ATCC). The Neuro-2A clone was obtained, from a spontaneous tumour originating from an A albino mouse strain, by R. J Klebe et al. This Neuro-2A line is subsequently stably transfected with 8NBRE-luciferase. The N2A-8NBRE cells are cultured until confluence in 75 cm² culture flasks containing DMEM supplemented with 10% of foetal calf serum, 4.5 g/l of glucose and 0.4 mg/ml of geneticin. After a week of culture, the cells are recovered with 0.25% trypsin for 30 seconds and then resuspended in DMEM without phenol red, containing 4.5 g/l of glucose and 10% of Hyclone delipidized serum, and deposited into transparent-bottom 96-well white plates. The cells are deposited at a rate of 60 000 per well in 75 μl for 24 hours before the addition of the products. The products are applied in 25 μl and incubated for a further 24 hours. On the day of the measurement, an equivalent volume (100 μl) of Steadylite is added to each well and then left for a period of 30 minutes in order to obtain complete cell lysis and maximum signal production. The plates are subsequently measured in a luminescence counter for microplates after having been sealed with an adhesive film. The products are prepared in the form of a stock solution at 10⁻²M and then diluted in 100% of DMSO. Each product concentration is prediluted in culture medium before incubation with the cells, thus containing 0.625% final concentration of DMSO.

For example, compounds Nos. 7, 8, 17, 30, 35 and 43 showed an EC₅₀ value of 5 nM, 0.8 nM, 6.7 nM, 56 nM, 11 nM and 2.4 nM respectively.

It is thus apparent that the compounds according to the invention have a modulatory effect on NOT.

The compounds chosen from the compounds of formula (I) as defined above, 2-(2,3-dihydro-1H-indol-1-ylcarbonyl)-5-methylimidazo[1,2-α]pyridine and 2-(4-thiomorpholin-1-ylcarbonyl)-6-chloroimidazo[1,2-α]pyridine, and the addition salts of these compounds with a pharmaceutically acceptable acid, can thus be used in the preparation of medicaments for their therapeutic application in the treatment or prevention of diseases involving NOT receptors.

Thus, according to another of its aspects, a subject-matter of the invention is a medicament which comprises a compound chosen from the compounds of formula (I) as defined above, 2-(2,3-dihydro-1H-indol-1-ylcarbonyl)-5-methylimidazo[1,2-α]pyridine and 2-(4-thiomorpholin-1-ylcarbonyl)-6-chloroimidazo[1,2-α]pyridine, and the addition salts of these compounds with a pharmaceutically acceptable acid, and more particularly which comprises a compound of formula (I) or one of its addition salts with a pharmaceutically acceptable acid.

These medicaments are employed therapeutically, in particular in the treatment and prevention of neurodegenerative diseases, such as, for example, Parkinson's disease, Alzheimer's disease or tauopathies (for example, progressive supranuclear palsy, frontotemporal dementia, corticobasal degeneration or Pick's disease); cerebral traumas, such as ischaemia and cranial traumas and epilepsy, psychiatric diseases, such as schizophrenia, depression, substance dependence or attention deficit hyperactivity disorders; inflammatory diseases of the central nervous system, such as multiple sclerosis, encephalitis, myelitis and encephalomyelitis, and other inflammatory diseases, such as vascular pathologies, atherosclerosis, inflammations of the joints, arthrosis or rheumatoid arthritis; osteoarthritis, Crohn's disease, ulcerative colitis; allergic inflammatory diseases, such as asthma; autoimmume diseases, such as type 1 diabetes, lupus, scleroderma, Guillain-Barré syndrome, Addison's disease and other immune-mediated diseases; osteoporosis; or cancers.

Thus, the present invention is targeted at a compound chosen from a compound of formula (I) as defined above, 2-(2,3-dihydro-1H-indol-1-ylcarbonyl)-5-methylimidazo[1,2-α]pyridine and 2-(4-thiomorpholin-1-ylcarbonyl)-6-chloroimidazo[1,2-α]pyridine, and the addition salts of these compounds with a pharmaceutically acceptable acid, in the treatment and prevention of one of the abovementioned diseases.

According to a specific embodiment, these medicaments are employed in the treatment and prevention of one of the abovementioned diseases, with the exception of cancers.

According to another of its aspects, the present invention relates to the use of a compound chosen from the abovementioned compounds in the preparation of a medicament intended for the treatment and prevention of one of the abovementioned diseases.

These compounds might also be used as treatment associated with stem cell transplants and/or grafts.

According to another of its aspects, the present invention relates to pharmaceutical compositions comprising, as active principle, a compound chosen from the group of the compounds defined above. These pharmaceutical compositions comprise an effective dose of at least one compound chosen from the group of the compounds defined above, or a pharmaceutically acceptable salt of the said compound, and also at least one pharmaceutically acceptable excipient.

The said excipients are chosen, depending on the pharmaceutical form and the method of administration desired, from the usual excipients which are known to a person skilled in the art.

In the pharmaceutical compositions of the present invention for oral, sublingual, subcutaneous, intramuscular, intravenous, topical, local, intratracheal, intranasal, transdermal or rectal administration, the active principle chosen from the group of the compounds defined above, or its salt, can be administered in unit administration form, as a mixture with conventional pharmaceutical excipients, to animals and human beings for the prophylaxis or treatment of the above disorders or diseases.

The appropriate unit administration forms comprise oral forms, such as tablets, soft or hard gelatin capsules, powders, granules and oral solutions or suspensions, forms for sublingual, buccal, intratracheal, intraocular or intranasal administration or for administration by inhalation, forms for topical, transdermal, subcutaneous, intramuscular or intravenous administration, forms for rectal administration and implants. For topical application, the compounds according to the invention can be used in creams, gels, ointments or lotions.

By way of example, a unit administration form of a compound according to the invention in the tablet form can comprise the following components:

| | |
|---|---|
| Compound according to the invention | 50.0 mg |
| Mannitol | 223.75 mg |
| Croscarmellose sodium | 6.0 mg |
| Maize starch | 15.0 mg |
| Hydroxypropylmethylcellulose | 2.25 mg |
| Magnesium stearate | 3.0 mg |

There may be specific cases where higher or lower dosages are appropriate; such dosages do not depart from the scope of the invention. According to the usual practice, the dosage appropriate to each patient is determined by the physician according to the method of administration and the weight and the response of the said patient.

The present invention, according to another of its aspects, also relates to a method for the treatment of the pathologies indicated above which comprises the administration, to a patient, of an effective dose of a compound chosen from the group of the compounds defined above or one of its pharmaceutically acceptable salts.

It is understood that all the subject-matters of the invention defined above, in particular medicament, pharmaceutical composition and treatment method, also relate more particularly to the subsets of compounds defined above.

What is claimed is:

1. A compound of formula (I): 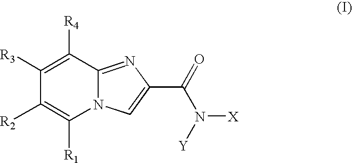
wherein:
X and Y form, with the nitrogen atom which carries them, a saturated or partially saturated, mono- or bicyclic, 5 to 10-membered cyclic amine optionally comprising from 1 to 4 additional heteroatoms chosen from O, S or N which is optionally substituted by a halogen atom or a (C₁-C₆)alkyl, (C₁-C₆)alkoxy, cyano, NRaRb or COOR₈ group, the said (C₁-C₆)alkyl and (C₁-C₆)alkoxy groups optionally being substituted by one or more halogen atoms;
R₁ represents a hydrogen atom, a halogen atom or a (C₁-C₆)alkoxy, (C₁-C₆)alkyl or NRcRd group, it being possible for the alkyl and alkoxy groups optionally to be substituted by one or more halogen atoms or a hydroxyl, amino or (C₁-C₆)alkoxy group;
R₂ represents one of the following groups:
a hydrogen atom,
a (C₁-C₆)alkyl group optionally substituted by one or more groups chosen, independently of one another, from a hydroxyl, a halogen atom, an NRaRb group, a (C₁-C₆)alkoxy group or a phenyl group,
a (C₁-C₆)alkoxy group optionally substituted by one or more groups chosen, independently of one another, from a hydroxyl, a halogen atom or an NRaRb group,
a (C₂-C₆)alkenyl group,
a (C₂-C₆)alkynyl group,
a —CO—R₅ group,
a —CO—NR₆R₇ group,
a —CO—O—R₈ group,
an —NR₉—CO—R₁₀ group,
an —N═CH—NRaRb group,
an NR₁₁R₁₂ group,
a halogen atom,
a cyano, nitro, hydroxyiminoalkyl or alkoxyiminoalkyl group,
a (C₁-C₆)alkylthio group,
a (C₁-C₆)alkylsulphinyl group,
a (C₁-C₆)alkylsulphonyl group,
a ((C₁-C₆)alkyl)₃silylethynyl group,
an —SO₂—NR₉R₁₀ group,
a phenyl group optionally substituted by one or more groups chosen, independently of one another, from the following atoms or groups: halogen, (C₁-C₆)alkoxy, cyano, NRaRb, —CO—R₅, —CO—NR₆R₇, —CO—O—R₈ or (C₁-C₆)alkyl optionally substituted by one or more hydroxyl or NRaRb groups, or
a heterocyclic group optionally substituted by a halogen atom or a (C₁-C₆)alkyl, (C₁-C₆)alkoxy, cyano, NRaRb or COOR₈ group, the (C₁-C₆)alkyl and (C₁-C₆)alkoxy groups optionally being substituted by one or more halogen atoms or hydroxyl groups;
R₃ represents a hydrogen atom, a (C₂-C₆)alkyl group, a (C₁-C₆)alkoxy group or a halogen atom;
R₄ represents a hydrogen atom, a (C₁-C₄)alkyl group, a (C₁-C₄)alkoxy group or a fluorine atom;
R₅ represents a hydrogen atom, a phenyl group or a (C₁-C₆)alkyl group;
R₆ and R₇, which are identical or different, represent a hydrogen atom or a (C₁-C₆)alkyl group or form, with the nitrogen atom which carries them, a 4- to 7-membered ring optionally including another heteroatom chosen from N, O or S;
R₈ represents a (C₁-C₆)alkyl group;
R₉ and R₁₀, which are identical or different, represent a hydrogen atom or a (C₁-C₆)alkyl group;
R₁₁ and R₁₂, which are identical or different, represent a hydrogen atom or a (C₁-C₆)alkyl group; and
Ra and Rb are, independently of one another, hydrogen or (C₁-C₆)alkyl or form, with the nitrogen atom which carries them, a 4- to 7-membered ring;
Rc is hydrogen and Rd is (C₁-C₆)alkyl;
or an acid addition salt thereof;
with the exception of 2-(2,3-dihydro-1H-indol-1-ylcarbonyl)-5-methylimidazo[1,2-α]pyridine and 2-(4-thiomorpholin-1-ylcarbonyl)-6-chloroimidazo[1,2-α]pyridine.

2. The compound of formula (I) according to claim 1, wherein R₂ is other than a hydrogen or chlorine atom;
or an acid addition salt thereof.

3. The compound of formula (I) according to claim 1, wherein:
R₂ represents one of the following groups:
a (C₁-C₆)alkoxy group,
an NR₁₁R₁₂ group,
a phenyl group optionally substituted by a group chosen from a —CO—R₅ or (C₁-C₆)alkyl group, itself optionally substituted by a hydroxyl; or
a heterocyclic group optionally substituted by an NRaRb group;
R₅ represents a (C₁-C₆)alkyl group;
R₁₁ and R₁₂, which are identical or different, represent a hydrogen atom or a (C₁-C₆)alkyl group; and
Ra and Rb are, independently of one another, hydrogen or (C₁-C₆)alkyl;
or an acid addition salt thereof.

4. The compound of formula (I) according to claim 1, wherein:
X and Y form, with the nitrogen atom which carries them, a saturated or partially saturated, mono- or bicyclic, 5- to 10-membered cyclic amine optionally comprising an additional heteroatom chosen from O or S and optionally substituted by a group chosen from a halogen atom;
or an acid addition salt thereof.

5. The compound of formula (I) according to claim 1, wherein:
-NKY represents a dihydrobenzoxazine, indoline, isoindoline, morpholine, piperidine, pyrrolidine, pyrroline, tetrahydropyridine, tetrahydroquinoline, thiomorpholine or tetrahydrothienopyridine group optionally substituted by a halogen atom;
R₂ represents one of the following groups:
a (C₁-C₆)alkoxy group,
an NR₁₁R₁₂ group,
a phenyl group optionally substituted by a group chosen from a halogen, a (C₁-C₆)alkoxy group or a (C₁-C₆)alkyl group optionally substituted by a hydroxyl or a C(O)R₅ group, or a pyridyl group, a pyrazolyl group, a furyl group, an oxazolyl group, a triazolyl group, a pyrrolyl group or an imidazolyl group optionally substituted by an NRaRb or (C₁-C₆)alkyl group, itself optionally substituted by a hydroxyl;
R₅ represents a (C₁-C₆)alkyl group;
R₁₁ and R₁₂, which are identical or different, represent a hydrogen atom or a (C₁-C₆)alkyl group;
Ra and Rb are, independently of one another, hydrogen or (C₁-C₆)alkyl; and
R₁, R₃ and R₄ represent a hydrogen atom;
or an acid addition salt thereof.

6. The compound of formula (I) according to claim 1, wherein:
NXY represents a dihydrobenzoxazine group, an indoline group optionally substituted by a fluorine atom, an isoindoline group, a morpholine group, a piperidine group, a pyrrolidine group, a pyrroline group, a tetrahydropyridine group, a tetrahydroquinoline group, a thiomorpholine group or a tetrahydrothienopyridine group;
R₂ represents a methoxy group, a phenyl group substituted by a hydroxymethyl, hydroxyethyl, hydroxymethylethyl, acetyl or N-dimethyl group, a pyridyl group optionally substituted by an amino group, a pyrazolyl group, a furyl group optionally substituted by a hydroxymethyl group, an oxazolyl group, a triazolyl group, a pyrrolyl group or an imidazoyl group;
R₁, R₃ and R₄ represent a hydrogen atom;
or an acid addition salt thereof.

7. The compound according to claim 1, selected from the group consisting of:
{3-[2-(Piperidin-1-ylcarbonyl)imidazo[1,2-α]pyridin-6-yl]phenyl}methanol;
{3-[2-(2,3-Dihydro-4H-1,4-benzoxazin-4-ylcarbonyl)imidazo[1,2-α]pyridin-6-yl]phenyl}methanol;
{3-[2-(Morpholin-4-ylcarbonyl)imidazo[1,2-α]pyridin-6-yl]phenyl}methanol;
2-(1,2,5,6-Tetrahydropyridin-1-yl)carbonyl-N,N-dimethylimidazo[1,2-α]pyridin-6-amine;
{3-[2-(2,3-Dihydro-1H-indol-1-ylcarbonyl)imidazo[1,2-α]pyridin-6-yl]phenyl}methanol;
(3-{2-[(6-Fluoro-2,3-dihydro-1H-indol-1-yl)carbonyl]imidazo[1,2-α]pyridin-6-yl}phenyl)methanol;
{3-[2-(2,5-Dihydro-1H-pyrrol-1-ylcarbonyl)imidazo[1,2-α]pyridin-6-yl]phenyl}methanol;
{3-[2-[(1,2,5,6-Tetrahydropyridin-1-yl)carbonyl]imidazo[1,2-α]pyridin-6-yl]phenyl}methanol;
{3-[2-(Pyrrolidin-1-ylcarbonyl)imidazo[1,2-α]pyridin-6-yl]phenyl}methanol;
{3-[2-(4,5,6,7-Tetrahydrothieno[3,2-α]pyridin-5-ylcarbonyl)imidazo[1,2-α]pyridin-6-yl]phenyl}methanol;
2-(2,3-Dihydro-4H-1,4-benzoxazin-4-ylcarbonyl)-N,N-dimethylimidazo[1,2-α]pyridin-6-amine;
{3-[2-(1,2,3,4-Tetrahydroquinoline-1-ylcarbonyl)imidazo[1,2-α]pyridin-6-yl]phenyl}methanol;
{3-[2-(1,3-Dihydro-2H-isoindol-2-ylcarbonyl)imidazo[1,2-α]pyridin-6-yl]phenyl}methanol;
2-(2,3-Dihydro-1H-indol-1-ylcarbonyl)-N,N-dimethylimidazo[1,2-α]pyridin-6-amine;
2-(1,3-Dihydro-2H-isoindol-2-ylcarbonyl)-N,N-dimethylimidazo[1,2-α]pyridin-6-amine;
N,N-Dimethyl-2-(piperidin-1-ylcarbonyl)imidazo[1,2-α]pyridin-6-amine;
2-[(6-Fluoro-2,3-dihydro-1H-indol-1-yl)carbonyl]-N,N-dimethylimidazo[1,2-α]pyridin-6-amine;
N,N-Dimethyl-2-(morpholin-4-ylcarbonyl)imidazo[1,2-α]pyridin-6-amine;
2-(2,5-Dihydro-1H-pyrrol-1-ylcarbonyl)-N,N-dimethylimidazo[1,2-α]pyridin-6-amine;
{3-[2-(Thiomorpholin-4-ylcarbonyl)imidazo[1,2-α]pyridin-6-yl]phenyl}methanol;
N,N-Dimethyl-2-(pyrrolidin-1-ylcarbonyl)imidazo[1,2-α]pyridin-6-amine;
2-(4,5,6,7-Tetrahydrothieno[3,2-c]pyridin-5-ylcarbonyl)-N,N-dimethylimidazo[1,2-α]pyridin-6-amine;
N,N-Dimethyl-2-(thiomorpholin-4-ylcarbonyl)imidazo[1,2-α]pyridin-6-amine;
2-(1,2,5,6-Tetrahydropyridin-1-yl)carbonyl-6-methoxy-5-methylimidazo[1,2-α]pyridine;
6-(Pyridin-2-yl)-2-[(1,2,5,6-tetrahydropyridin-1-yl)carbonyl]imidazo[1,2-α]pyridine;
2-(2,3-Dihydro-1H-indol-1-ylcarbonyl)-6-(pyridin-2-yl)imidazo[1,2-α]pyridine;
2-(2,3-Dihydro-1H-indol-1-ylcarbonyl)-6-(1H-pyrazol-3-yl)imidazo[1,2-α]pyridine;
6-(Furan-2-yl)-2-[(1,2,5,6-tetrahydropyridin-1-yl)carbonyl]imidazo[1,2-α]pyridine;
2-(2,3-Dihydro-1H-indol-1-ylcarbonyl)-6-(furan-2-yl)imidazo[1,2-α]pyridine;
6-(Oxazol-2-yl)-2-[(1,2,5,6-tetrahydropyridin-1-yl)carbonyl]imidazo[1,2-α]pyridine;
6-(Furan-3-yl)-2-[(1,2,5,6-tetrahydropyridin-1-yl)carbonyl]imidazo[1,2-α]pyridine;
2-(2,3-Dihydro-1H-indol-1-ylcarbonyl)-6-(furan-3-yl)imidazo[1,2-α]pyridine;
6-[5-(Hydroxymethyl)furan-2-yl]-2-[(1,2,5,6-tetrahydropyridin-1-yl)carbonyl]imidazo[1,2-α]pyridine;
[(1,2,5,6-Tetrahydropyridin-1-yl)carbonyl]-6-(1H-1,2,4-triazol-3-yl)imidazo[1,2-α]pyridine;
2-(2,3-Dihydro-1H-indol-1-ylcarbonyl)-6-(1H-1,2,4-triazol-3-yl)imidazo[1,2-α]pyridine;
6-(6-Aminopyridin-2-yl)-2-[(1,2,5,6-tetrahydropyridin-1-yl)carbonyl]imidazo[1,2-α]pyridine and its dihydrochloride;
6-(6-Aminopyridin-2-yl)-2-(2,3-Dihydro-1H-indol-1-ylcarbonyl)imidazo[1,2-α]pyridine and its dihydrochloride;
6-(1H-Pyrrol-3-yl)-2-[(1,2,5,6-tetrahydropyridin-1-yl)carbonyl]imidazo[1,2-α]pyridine;
2-(2,3-Dihydro-1H-indol-1-ylcarbonyl)-6-(1H-pyrrol-3-yl)imidazo[1,2-α]pyridine;
2-(2,3-Dihydro-1H-indol-1-ylcarbonyl)-6-(furan-2-yl)imidazo[1,2-α]pyridine and its trifluoroacetate(1•1);
2-(2,3-Dihydro-1H-indol-1-ylcarbonyl)-6-[5-(hydroxymethyl)furan-2-yl]imidazo[1,2-α]pyridine and its trifluoroacetate(1•1);
6-(1H-Imidazol-4-yl)-2-[(1,2,5,6-tetrahydropyridin-1-yl)carbonyl]imidazo[1,2-α]pyridine;
2-(2,3-Dihydro-1H-indol-1-ylcarbonyl)-6-(1H-imidazol-4-yl)imidazo[1,2-α]pyridine;
2-[(1,2,5,6-Tetrahydropyridin-1-yl)carbonyl]-6-(1H-1,2,3-triazol-4-yl)imidazo[1,2-α]pyridine;
2-(2,3-Dihydro-1H-indol-1-ylcarbonyl)-6-(1H-1,2,3-triazol-4-yl)imidazo[1,2-α]pyridine;
6-(3-Acetylphenyl)-2-[(1,2,5,6-tetrahydropyridin-1-yl)carbonyl]imidazo[1,2-α]pyridine;
(RS)-6-[3-(1-Hydroxyethyl)phenyl]-2-[(1,2,5,6-tetrahydropyridin-1-yl)carbonyl]imidazo[1,2-α]pyridine; and
6-[3-(1-Hydroxy-1-methylethyl)phenyl]-2-[(1,2,5,6-tetrahydropyridin-1-yl)carbonyl]imidazo[1,2-α]pyridine.

8. A pharmaceutical composition comprising a compound according to claim 1, or 7,2-(2,3-dihydro-1H-indol-1-ylcarbonyl)-5-methylimidazo[1,2-α]pyridine or 2-(4-thiomorpholin-1-ylcarbonyl)-6-chloro-imidazo[1,2-α]pyridine, or a pharmaceutically acceptable salt of these compounds; and also at least one pharmaceutically acceptable excipient.

9. A pharmaceutical composition comprising a compound according to claim 1, or 7,2-(2,3-dihydro-1H-indol-1-ylcarbonyl)-5-methylimidazo[1,2-α]pyridine or 2-(4-thiomorpholin-1-ylcarbonyl)-6-chloro-imidazo[1,2-α]pyridine, or a pharmaceutically acceptable salt of these compounds; and also at least one pharmaceutically acceptable excipient.

10. A method of treating or preventing a disease comprising administering to a patient an effective amount of a compound according to claim 1, or 7,2-(2,3-dihydro-1H-indol-1-ylcarbonyl)-5-methylimidazo[1,2-α]pyridine or 2-(4-thiomorpholin-1-ylcarbonyl)-6-chloro-imidazo[1,2-α]pyridine; or a pharmaceutically acceptable salt thereof.

11. The method according to claim 10, wherein the disease is a neurodegenerative disease.

12. The method according to claim 10, wherein the disease is cerebral trauma or epilepsy.

13. The method according to claim 10, wherein the disease is a psychiatric disease.

14. The method according to claim 10, wherein the disease is an inflammatory disease.

15. The method according to claim 10, wherein the disease is osteoporosis.

16. The method according to claim 10, wherein the disease is cancer.

17. The method according to claim 10, wherein the disease is Parkinson's disease, Alzheimer's disease, tauopathies or multiple sclerosis.

18. The method according to claim 10, wherein the disease is schizophrenia, depression, substance dependence or attention deficit hyperactivity disorder.

19. A compound selected from the group consisting of:
Ethyl 6-methoxy-5-methylimidazo[1,2-α]pyridine-2-carboxylate;
6-Methoxy-5-methylimidazo[1,2-α]pyridine-2-carboxylic acid;
Ethyl 6-[3-(Hydroxymethyl)phenyl]imidazo[1,2-α]pyridine-2-carboxylate;
6-[3-(Hydroxymethyl)phenyl]imidazo[1,2-α]pyridine-2-carboxylic acid;
Ethyl 6-(6-{[(1,1-dimethylethoxy)carbonyl]amino}pyridin-2-yl)imidazo[1,2-α]pyridine-2-carboxylate;
6-(6-{[(1,1-Dimethylethoxy)carbonyl]amino}pyridin-2-yl)imidazo[1,2-α]pyridine-2-carboxylic acid;
Ethyl 6-(Pyridin-2-yl)imidazo[1,2-α]pyridine-2-carboxylate; 6-(Pyridin-2-yl)imidazo[1,2-α]pyridine-2-carboxylic acid;
Ethyl 6-(1-triphenylmethyl-1H-imidazol-4-yl)imidazo[1,2-α]pyridine-2-carboxylate;
6-(1-triphenylmethyl-1H-imidazol-4-yl)imidazo[1,2-α]pyridine-2-carboxylic acid;
Ethyl 6-[1-(triisopropylsilyl)-1H-pyrrol-3-yl]imidazo[1,2-α]pyridine-2-carboxylate;
6-(1H-Pyrrol-3-yl)imidazo[1,2-α]pyridine-2-carboxylic acid;
Ethyl 6-(1H-pyrazol-3-yl)imidazo[1,2-α]pyridine-2-carboxylate;
6-(1H-pyrazol-3-yl)imidazo[1,2-α]pyridine-2-carboxylic acid;
Ethyl 6-(furan-2-yl)imidazo[1,2-α]pyridine-2-carboxylate;
6-(Furan-2-yl)imidazo[1,2-α]pyridine-2-carboxylic acid;
Ethyl 6-(furan-3-yl)imidazo[1,2-α]pyridine-2-carboxylate;
6-(Furan-3-yl)imidazo[1,2-α]pyridine-2-carboxylic acid;
Ethyl 6-[5-(hydroxymethyl)furan-2-yl]imidazo[1,2-α]pyridine-2-carboxylate;
6-[5-(Hydroxymethyl)furan-2-yl]imidazo[1,2-α]pyridine-2-carboxylic acid;
Ethyl 6-(oxazol-2-yl)imidazo[1,2-α]pyridine-2-carboxylate;
6-(Oxazol-2-yl)imidazo[1,2-α]pyridine-2-carboxylic acid;
Ethyl 6-(1H-1,2,4-triazol-3-yl)imidazo[1,2-α]pyridine-2-carboxylate;
6-(1H-1,2,4-Triazol-3-yl)imidazo[1,2-α]pyridine-2-carboxylic acid;
Ethyl 6-(1H-1,2,3-triazol-4-yl)imidazo[1,2-α]pyridine-2-carboxylate; and
6-(1H-1,2,3-Triazol-4-yl)imidazo[1,2-α]pyridine-2-carboxylic acid.
